(19) United States
(12) Patent Application Publication
(10) Pub. No.: US 2011/0160123 A1
(43) Pub. Date: Jun. 30, 2011

(54) TARGETING THE EFFLUX SYSTEMS OF MYCOBACTERIUM TUBERCULOSIS

(75) Inventor: Michael Niederweis, Homewood, AL (US)

(73) Assignee: THE UAB RESEARCH FOUNDATION, Birmingham, AL (US)

(21) Appl. No.: 12/993,649

(22) PCT Filed: May 20, 2009
(86) PCT No.: PCT/US09/44707

§ 371 Date: Nov. 29, 2010

Related U.S. Application Data
(60) Provisional application No. 61/054,759, filed on May 20, 2008.

(51) Int. Cl. A61K 38/02; A61K 31/44; A61K 31/7088; C12Q 1/18; C12Q 1/68; A61P 31/06

(52) U.S. Cl. 514/2.4; 514/358; 514/44 R; 435/32; 435/6.1

(57) ABSTRACT

Provided herein are methods of reducing drug resistance in Mycobacterium tuberculosis (Mtb). The methods comprise contacting the Mtb with an agent, wherein the agent inhibits the activity of an efflux complex. Also provided are methods of treating Mtb in a subject. The methods comprise administering to the subject an agent that inhibits the activity of an efflux complex; and administering to the subject a tuberculosis treating agent. Further provided is a method of screening for an agent that reduces drug resistance in Mtb. The methods comprise providing an Mtb with a mutant efflux complex; and contacting Mtb with an agent to be tested and a tuberculosis treating agent.

### BACKGROUND

Mycobacterium tuberculosis (Mtb) has infected about two billion people and causes the death of about two million people every year, more than any other pathogenic bacterium. Since the lungs of an infected patient contain more than a billion bacilli, poor treatment compliance selects for multi drug resistant (MDR) strains. Mtb is intrinsically resistant to many drugs mainly due to an impermeable outer membrane (OM) in combination with the activities of multidrug efflux pumps. All current first and many second line Tuberculosis (TB) drugs are substrates of one or several drug efflux pumps. TB caused by Mtb strains resistant to the few available TB drugs increases both the treatment time and the cost of treatment dramatically.

### SUMMARY

Provided herein are methods of reducing drug resistance in Mycobacterium tuberculosis (Mtb). The methods comprise contacting Mtb with an agent, wherein the agent inhibits the activity of an efflux complex.

Also provided are methods of treating Mtb in a subject. The methods comprise administering to the subject an agent that inhibits the activity of an efflux complex and administering to the subject a tuberculosis treating agent.

Further provided is a method of screening for an agent that reduces drug resistance in Mtb. The methods comprise providing an Mtb with a mutant efflux complex and contacting the Mtb with an agent to be tested and a tuberculosis treating agent. Reduced resistance to the tuberculosis treating agent in the presence of the agent to be tested, as compared to a control, indicates the agent to be screened reduces drug resistance in Mtb.

### DESCRIPTION OF DRAWINGS

FIG. 1 shows a bar graph demonstrating that expression of rv1698 increases the antibiotic susceptibility of the M. smegmatis porin mutant MN01. The susceptibilities of M. smegmatis wild type with the control vector pMS2 (white bars), the ΔmspA mutant MN01 with the control vector pMS2 (black bars), MN01 with the mspA expression vector pMN014 (light gray bars), and MN01 with the rv1698 expression vector pMN035 (dark gray bars) to 32 μg/ml ampicillin (Amp), 3 μg/ml cephaloridine (Ceph), and 6 μg/ml chloramphenicol (Chl) were determined by agar dilution on 7H10 Middlebrook agar plates containing 2% glycerol. The number of colonies on the antibiotic plates was normalized to the number of colonies on plates without antibiotic for each strain and expressed as relative colony-forming units (% cfu).

FIG. 2 shows a graph demonstrating the Rv1698-dependent glucose uptake by a porin mutant of M. smegmatis. Accumulation of [¹⁴C]glucose by the ΔmspA ΔmspC double mutant M. smegmatis ML10 containing the empty vector pMS2 (filled circles, control), the mspA expression vector pMN014 (filled squares) and the rv1698 expression vector pMN035 (open circles). Both genes are transcribed from the p_{smyc} promoter. The assay was performed at 37° C. at a final glucose concentration of 20 μM. The uptake experiment was done in triplicate and is shown with standard deviations.

FIG. 3 shows single channel recordings of purified recombinant Rv1698_{His} in lipid bilayer experiments. FIG. 3A shows an image of a silver stained gel demonstrating expression and purification of recombinant Rv1698_{His}. The rv1698 gene was expressed in the E. coli Rosetta strain using the plasmid pML 122. The parent plasmid pET-28b+does not contain the rv1698 gene and was used as a control. The samples were separated on a 10% polyacrylamide gel stained with silver. Lane M, molecular mass marker; lane 1, unsoluble fraction of E. coli Rosetta with pET-28b+; lanes 2 and 3, unsoluble fractions of E. coli Rosetta with pML122 before (lane 2) and after (lane 3) induction with isopropylthio-β-_{D}-galactosidase; lane 4, unsoluble fraction (lane 3) dissolved in 8 M urea; lane 5, flow through from Ni²⁺ column; lane 6, elution at pH 6.3; lane 7, elution at pH 4.5; lane 8, purified Rv1698 after dilution in Na—P buffer with 0.5% OPOE. FIG. 3B shows a histogram demonstrating single channel recordings of purified Rv1698_{His} in lipid bilayer experiments. The current intensity corresponding to the insertion of single channels inside a DPhPC membrane bathing in 1 M KCl was recorded after the addition of 30 ng of purified rRv1698_{His} to both sides of the membrane (final concentration, 3 ng/ml). The data were collected from seven different membranes. The most frequent insertions had a single channel conductance of 4.5 nS.

FIGS. 4A through 4F show the analysis of the ion specificity of rRv1698_{His}. The single channel conductance of rRv1698_{His} was determined in different electrolytes. The concentration of each electrolyte was 1 M. The probability P of a conductance step G was calculated from 46 (KCl) (FIGS. 4A and 4D), 27 (NaCl) (FIG. 4B), 48 (LiCl) (FIG. 4C), 70 (KNO3) (FIG. 4E), and 80 (KAc) (FIG. 4F) insertion events from five to seven membranes. The panels on the left and right sides show the change of the conductance of Rv1698_{His} in dependence on the size of the cation and anions, respectively. The reference electrolyte is KCl and is shown on both sides of this figure for comparison purposes. Thus, A and D are identical.

FIG. 5 shows an image of gel demonstrating the analysis of expression of rv1698 in M. tuberculosis. The RT-PCR products were separated on a 1% agarose gel. The length of the product is 400 bp. The sample, in which the reverse transcriptase was added for the cDNA synthesis, is marked with +, whereas the − sign denotes the sample in which reverse transcriptase was omitted to detect contaminations with chromosomal DNA. DNA denotes samples where chromosomal DNA was used as a template for the PCR to analyze the specificity of the primers. The gel was stained with ethidium bromide and is shown as a negative image to enhance the visibility of weak bands.

FIG. 6 shows the overexpression and purification of Rv1698_{His} from M. bovis BCG and M. smegmatis. The proteins were extracted from M. bovis BCG/pML911 and M. smegmatis SMR5/pML911 lysates using 1% SDS in PBS. Rv1698_{His} proteins were purified from these extracts by Ni²⁺ affinity chromatography. All of the samples were analyzed on 10% polyacrylamide gels. FIG. 6A shows an image of a Coomassie-stained gel. 5 μg of protein in raw extracts and 50 ng of the purified Rv1698_{His} proteins were loaded. FIG. 6B shows an image of a Western blot demonstrating expression of Rv1698_{His}. 5 μg of protein in raw extracts and 10 ng of the purified Rv1698_{His} proteins were loaded. The proteins were transferred onto a polyvinylidene difluoridemembrane and detected using an Rv1698-specific polyclonal antiserum. Lane 1, raw extract from M. bovis BCG/pML911; lane 2, purified Rv1698_{His} from M. bovis BCG/pML911; lane 3, raw extract from M. smegmatis SMR5/pML911; lane 4, purified Rv1698_{His} from M. smegmatis SMR5/pML911; lane M, molecular mass marker. The Rv1698_{His} monomer and its putative dimer are marked with M and D, respectively.

FIG. 7 shows the single channel activity of Rv1698_{His} purified from M. bovis BCG. Rv1698_{His} protein was purified by Ni²⁺ affinity chromatography from 0.5% OPOE extracts obtained from M. bovis BCG/pML911. Purified Rv1698_{His} protein was added to the cis-side of a DPhPC membrane bathed by 1MKCl, 10 mMHEPES, pH7.0, and a −10 mV potential was applied. The channel activity was recorded using a data acquisition card. The boxed traces highlight opening and closing events of different sizes. FIG. 7A shows a histogram demonstrating the current trace for purified Rv1698_{His}. The current trace shows more than 30 opening and closing events recorded 600 seconds after addition of 300 ng of Rv1698_{His} to the membrane. The total recording time was 1843 seconds. FIG. 7B shows a histogram demonstrating the opening and closing events for the purified Rv1698_{His}. The histogram represents a total of 109 opening and closing events recorded from seven membranes. FIG. 7C shows a histogram demonstrating a current trace for the purified Rv1698_{His}. The current trace shows more than eight opening and closing events recorded 400 seconds after the addition of 1 μg of Rv1698_{His} to the membrane. The total recording time was 2862 seconds.

FIG. 8 shows an image of a Western blot demonstrating the surface accessibility of Rv1698 in M. smegmatis by digestion with proteinase K. Whole cells of M. smegmatis were treated with proteinase K (+) or with PBS as a control (−). After adding protease inhibitors, the cells were washed in PBS buffer, and proteins were extracted with SDS by boiling. The solubilized proteins were analyzed in a 10% polyacrylamide gel and transferred to a polyvinylidene difluoride membrane. The proteins on these blots were specifically detected using the appropriate antibodies. M, molecular mass marker. The samples were extracts from M. smegmatis containing the plasmids pMN437 (green fluorescent protein (Gfp)), pMV61015.1 (PE_PGRS33HA), pML451 (Msmeg_{—}3747_{His}), and pML911 (Rv1698_{His}).

FIG. 9 shows the genomic region of the mutant M. bovis BCG ML1034 and its corresponding region in M. tuberculosis H37Rv. The bcg0231 gene and its flanking genes are depicted. Block arrows represent open reading frames. A vertical arrow depicts the insertion of the transposon 096::Km. The sequence of the DNA −200 to +18 (SEQ ID NO:1) relative to the rv0194/bcg0231 start codon is shown. This sequence is identical in M. tuberculosis H37Rv and M. bovis BCG. The black arrow depicts the start of the rv0194 gene with the potential start codon ATG. Putative Shine-Dalgarno and extended −10 promoter sequences are shown in bold and underlined, respectively. The annotated functions of the encoded proteins are as follows: Bcg0230c, hypothetical protein; Bcg0231, probable drug transport transmembrane ATP-binding protein cassette transporter; Bcg0232, possible two-component transcriptional regulatory protein; Bcg0233, possible transcriptional regulatory protein.

FIG. 10 shows a bar graph demonstrating the susceptibility of M. bovis BCG ML1034 to ampicillin. The susceptibilities of wild type (wt) M. bovis BCG (black bars) and of the ML1034 mutant (white bars) to ampicillin were determined by the microplate Alamar blue assay in triplicates. The percentage of survival is shown with standard deviations.

FIG. 11 shows bcg0231 mRNA levels are increased in the ML1034 mutant of M. bovis BCG. FIG. 11A shows an image of a dot blot experiment demonstrating that bcg0231 mRNA expression is increased in the ML1034 mutant. Total RNA was prepared from M. bovis BCG cultures in late logarithmic phase. A 7.2-μg sample of RNA was spotted onto duplicate membranes in triplicate. The bcg0231 mRNA and the 16S rRNA were detected using digoxigenin-labeled probes which were visualized with an anti-digoxigenin antibody-alkaline phosphatase conjugate and a chemiluminescent substrate. FIG. 11B shows a bar graph quantifying the level of bcg0231 expression in the dot blots. The chemiluminescence of the dots was quantified using integrative optical analysis. The lane profile of the dots was analyzed to examine saturation of the signals. The amount of bcg0231 transcripts was normalized to that of 16S rRNA in the same sample. The bcg0231 amounts detected for the ML1034 mutant were set as 100%.

FIG. 12 shows a bar graph demonstrating β-lactamase activity of wild-type M. bovis BCG and the ML1034 mutant. Hydrolysis of nitrocefin by whole-cell lysates (black bars) and culture filtrates (grey bars) was measured as absorption at 490 nm. The β-lactamase activity is shown as A₄₉₀ per min and mg of total protein. The background activity was determined using PBS as a negative control. All assays were performed in triplicate. Error bars represent standard deviations.

FIG. 13 shows the effects of rv1094 expression in M. smegmatis on accumulation of and killing by ethidium bromide. FIG. 13A shows a graph demonstrating accumulation of ethidum bromide by M. smegmatis over time. Accumulation of 20 μM ethidium bromide by M. smegmatis SMR5 transformed with control plasmid pMS2 (closed circles) and with the rv0194 expression vector pML655 (open triangles) was measured by fluorescence. A 0.1 mM solution of reserpine was added to half of the culture of SMR5/pML655 after 8 minutes of incubation with ethidium bromide (closed squares). Fluorescence was measured as relative fluorescence units (RFU) at an excitation wavelength of 530 nm and an emission wavelength of 590 nm. FIG. 13B shows a graph demonstrating the growth of M. smegmatis over time. Growth of M. smegmatis SMR5 transformed with control plasmid pMS2 (closed circles) and with the rv0194 expression vector pML655 (closed triangles) was measured in the presence of 1.56 mM ethidium bromide. Reserpine at a final concentration of 8 mM was added to cultures of M. smegmatis SMR5 with a control plasmid pMS2 (open circles) or with the rv0194 expression vector pML655 (open triangles) containing 1.56 mM ethidium bromide.

FIG. 14 shows the analysis of the ms3747 mutant of M. smegmatis. FIG. 14A shows an image of a Western blot demonstrating the expression of Ms3747 in M. smegmatis. Proteins were extracted with 2% SDS from wt M. smegmatis, the Δms3747 mutant ML77, and ML77 complemented with the ms3747 expression vector pML451. The proteins were detected in a Western blot using the monoclonal antibody 5D1.23 FIG. 14B shows images of colonies of the wt M. smegmatis and ML77 strain containing the control vector pMS2. FIG. 14C shows images of colonies of the ML77 strain complemented with the ms3747 expression vector and rv1698 expression vector.

FIG. 15 shows a bar graph demonstrating the accumulation of copper by the M. smegmatis ms3747 mutant. M. smegmatis SMR5 (black bars) and the ΔmctB mutant ML77 (grey bars) were grown in self-made 7H9 medium with 0, 6.3 or 25 μM CuSO₄. Samples of three independent cultures were taken after growth for 36 hours. Copper was determined by measuring the absorption of the Cu²′(dithizone)₂ complex at 533 nm.

FIG. 16 shows an image of a Western blot demonstrating that MctB (Rv1698) is not produced by the mctB mutant of M. tuberculosis. Proteins were extracted with 2% SDS from wt Mtb, the mctB mutant ML256 and ML256 complemented with the mctB expression vector pMN035. ML257 is a ML256 derivative carrying the integrative rv1698 expression vector pML955. The proteins were detected in a Western blot using the MctB monoclonal antibody 5D1.23.

FIG. 17 shows images of Mtb colonies demonstrating that MctB is required for copper efflux by M. tuberculosis. Pictures were taken of drops of two dilutions of cultures of Mtb H37Rv, ML256 (Δrv1698) and the complemented strain ML257 on 7H11/OADC plates. CuSO₄ was added at 150 μM. Bathocuproine disulfonate (BCS) binds Cu(I) and protects Mtb from the toxic effects of CuSO₄. Plates were incubated at 37° C. for 22 days (magnification:10×).

FIG. 18 shows a bar graph demonstrating that MctB is required for copper efflux by M. smegmatis. Mtb (black bars) and the Δrv1698 mutant ML256 (grey bars) were grown in HdB medium with 1.5, 6.3 or 25ₘM CuSO₄. Samples of three independent cultures were taken after growth for 10 days. Cellular copper was determined by measuring the absorption of the Cu²⁺ (dithizone)₂ complex at 533 nm.

FIG. 19 shows the role of the copper efflux channel MctB for virulence of Mtb in mice. BALB/c mice were infected with aerosols of wild-type Mtb H37Rv and the mctB mutant ML256. The colony forming units (cfu) were determined by plating lung homogenates from four mice and are shown with their standard deviations. FIG. 19A shows a graph demonstrating the cfu counts in the lungs of infected mice for both strains of Mtb. FIG. 19B shows a graph demonstrating the effect of 118 mg/L CuSO₄ in the drinking water on the persistence of both Mtb bacterial strains over time.

FIG. 20 shows a model of the efflux of drugs and Cu+ across the mycobacterial cell envelope. Covalent bonds between mycolic acids, arabinogalactan (AG) and peptidoglycan (PG) are indicated. Rv1698 is an OM channel required for copper efflux. CtpV is a putative IM efflux protein of Mtb for copper.

FIG. 21 shows images of a screen of a transposon library of M. smegmatis for mutants hypersusceptible to multiple antibiotics. 398 clones obtained from the initial screen on chloramphenicol were screened further using chloramphenicol (8 μg/mL), ampicillin (16 μg/mL), erythromycin (0.5 μg/mL), and norfloxacin (0.6 μg/mL). These concentrations were determined experimentally as sub-inhibitory for wt M. smegmatis using the same assay. Growth of the transposon mutants was monitored every four hours over three days. Plates containing only wt M. smegmatis were used as controls. Pictures of the plates shown were taken after incubation at 37° C. for 57 hours. 27 clones were hypersusceptible to all four antibiotics. Three of these clones are indicated by the circles on the plates. The plate containing chloramphenicol is not shown. An additional criterium for selecting hypersusceptible clones was a slower colony growth compared to other clones based on a kinetic analysis.

FIG. 22 shows an image of a Western blot demonstrating crosslinking of MctB_{Mtb} in M. smegmatis. M. smegmatis ML77 expressing MctB_{MtbHis} was treated with crosslinkers. MctB was detected with the monoclonal antibody 8A6-14 in a Western blot. Lanes: (M) molecular mass marker; (1) untreated cells; (2) 1% formaldehyde; (3) 1% formaldehyde, heated for 30 minutes at 100° C.; (4) 5 mM Dithiobis (succinimidyl) propionate (DSP); (5) 5 mM DSP, heated for 30 minutes at 100° C. in the presence of 4% β-mercaptoethanol. MctB_{MtbHis} crosslinked to other proteins is marked by stars. These complexes will be purified by Ni²⁺-affinity chromatography and sequenced by mass fingerprinting using MALDI-TOF. m: MctB monomer; d: dimer. Dimer formation was shown by sequence analysis by mass fingerprinting. Putative crosslinks of MctB to other proteins are marked by stars.

### DETAILED DESCRIPTION

Efficient drug efflux systems in gram-negative bacteria share a common outer membrane channel protein. Elimination of the outer membrane (OM) component increases the susceptibility to most drugs and helps to prevent the emergence of antibiotic resistance in other bacteria. Mycobacteria also have an outer membrane. Inhibition of the OM component in Mtb is useful to inactivate multi-drug efflux systems directly without the need to cross the OM permeability barrier (“channel blocker”). This avoids permeability problems and reduces the frequency of resistance mutations in Mtb. Thus, such OM protein inhibitors enable the use of established, but so far inefficient, antibiotics for TB chemotherapy.

The genome of Mtb encodes 69 putative drug efflux pumps. See De Rossi et al., FEMS Microbiol. Rev. 30:36-52 (2006), which is incorporated by reference herein at least for the putative drug efflux pumps and related methods. Drug efflux is essential in other bacteria for the emergence of antibiotic resistance. Only efflux of drugs across both membranes is an effective resistance mechanism in gram-negative bacteria. This process is mediated by tripartite efflux systems consisting of an inner membrane pump, an OM channel protein, and periplasmic adapter proteins.

Rv1698 is an OM channel protein of Mtb. A mutant of M. smegmatis lacking the Rv1698 homologue is very sensitive to copper because it accumulates 10-fold more copper than the wild-type. These and other results show that Rv1698 is part of a copper efflux system. Metal and drug-efflux systems share the same tripartite architecture in gram-negative bacteria.

Tap, Rv1634, Rv1258c and Stp of the Major Facilitator Superfamily (MFS) confer resistance to aminoglycosides, tetracycline, fluoroquinolones, rifampicin, ofloxacin, spectinomycin and tetracycline, respectively. MmR of the Small Multidrug Resistance (SMR) family provides resistance to different antiseptics, drugs, and intercalating dyes). The ATP-Binding Cassette (ABC) transporters DrrAB and Rv2686c-2687c-2688c confer resistance to hydrophobic drugs and to fluoroquinolones, respectively. The genome of Mtb also encodes 15 putative transporters of the Resistance, Nodulation and Cell Division (RND) family called MmpL proteins. It was shown that MmpL7 is indeed a drug efflux pump and provides high resistance to isoniazid.

Efflux pumps are inner membrane transporter proteins which use energy (ATP hydrolysis or proton gradient) to export solutes either into the periplasm or into the medium. The majority of the efflux pumps of Gram-negative bacteria connect to an OM protein and are especially effective, because they traverse both membranes and pump out drugs directly into the external medium. For example, transporters of the MFS, the ABC superfamily, and the RND superfamily require an OM channel for function. AcrB of E. coli belongs to the RND family and is one of the best examined efflux pumps. AcrB is part of a tripartite system consisting also of the OM channel TolC and the membrane fusion protein AcrA, which is anchored in the inner membrane by an N-terminal lipid moiety. Deletion of TolC alone increased the susceptibility of E. coli to multiple drugs in a manner similar to AcrAB.

TolC is an important, low-abundance protein in the OM of gram-negative bacteria. Although TolC of E. coli and its homologs such as OprM of P. aeruginosa share only little sequence similarities (40%), they have similar structures and functions. Planar lipid bilayer experiments showed that TolC and its homologs form water-filled channels with similar levels of conductance (approximately 80 pS in 1M KCl). Crystallography revealed that TolC and OprM share the same homo-trimeric structure, which spans the OM and periplasm of these bacteria as a channel-tunnel. These trimers form a 12-stranded β-barrel that lodges in the OM and a coiled α-helical barrel that spans the periplasm and forms a complex with inner membrane transport proteins such as AcrB of E. coli. The presence of the membrane fusion proteins AcrA/MexA appears to be required for opening of the tunnel. TolC functions as a component of multi-drug resistance (MDR) efflux systems in the removal of a broad range of toxic chemicals from the cell. Type I-dependent secretion of certain virulence-associated proteins also requires TolC. TolC is important for virulence and survival in the host of the pathogenic E. coli, Vibrio cholerae, Salmonella enterica serovar Enteritidis, and Serratia marcescens.

Mycobacteria produce mycolic acids which are α-branched β-hydroxy fatty acids consisting of up to 90 carbon atoms and the longest fatty acids known in nature. In addition, the mycobacterial cell envelope contains a fascinating diversity of other lipids, many of which are unique to mycobacteria. Minnikin originally proposed that the mycolic acids, which are covalently bound to the arabinogalactan-peptidoglycan co-polymer, form the inner layer of an unique OM (Minnikin, Lipids: Complex lipids, their chemistry, biosynthesis and roles. In: Ratledge, C., and Stanford, J. (eds). The biology of the mycobacteria: Physiology, identification and classification, Academic Press, London (1992)). Experimental evidence for this model was provided by X-ray diffraction studies, which showed that the mycolic acids are oriented in parallel and perpendicular to the plane of the cell envelope. The analysis of spin-labeled fatty acids inserted into isolated mycobacterial cell walls by electron paramagnetic resonance supported the existence of a moderately fluid outer leaflet while the inner leaflet consisting of mycolic acids has an extremely low fluidity. This interpretation is consistent with observations that mutants with defects in the production of some of the major extractable lipids (glycopeptidolipids, phthiocerol dimycocerosate) showed an increased OM permeability to hydrophobic solutes. Thus, the mycobacterial OM constitutes a supported asymmetric lipid bilayer and provides an extraordinarily efficient permeability barrier, which is 100-1,000-fold less permeable than that of E. coli.

The discovery of the MspA porin of M. smegmatis showed that OM proteins exist in mycobacteria and that they fulfil essential transport functions such as diffusion of small and hydrophilic nutrients in the case of porins. OM proteins are also needed for other transport processes such as multidrug efflux or protein secretion. The X-ray analysis of crystals revealed that the structure of MspA is completely different from those of porins of gram-negative bacteria.

Provided herein are methods of reducing drug resistance in Mycobacterium tuberculosis (Mtb). The methods comprise contacting the Mtb with an agent, wherein the agent inhibits the activity of an efflux complex. The efflux complex, for example, comprises an efflux channel and an efflux pump. The efflux channel can comprise Rv1698 (MctB) or a TolC-like efflux channel. The efflux pump can comprise Rv0194. The efflux complex can comprise a TolC-like efflux channel and Rv0194.

Optionally, the agent is selected from a group consisting of a small molecule, a polypeptide, a nucleic acid, or a peptidomimetic. The agent can, for example, inhibit the activity of the efflux channel. Optionally, the agent is an efflux channel inhibitor or blocker. Optionally, the efflux channel inhibitor or blocker comprises Ru(II)quaterpyridinium complex or a derivative thereof. The agent can, for example, inhibit the activity of the efflux pump.

Also provided herein are methods of treating or preventing Mycobacterium tuberculosis (Mtb) in a subject. The methods comprise administering to the subject an agent that inhibits the activity of an efflux complex and administering to the subject a tuberculosis treating agent. The tuberculosis treating agent, for example, comprises an antibiotic (e.g., isoniazid, ethambutol, rifampicin, norfloxacin, erythromycin, pyrazinamide, capreomycin, kanamycin, chloramphenicol, tetracycline, streptomycin, and vancomycin), or it comprises derivatives thereof (e.g., derivatives of penicillin, cephalosporin, macrolide, tetracycline, fluoroquinolone, nitroimidazole, aminoglycoside, sulfonamide, monobactams, carbapenems classes or rifampicin, diarylquinoline, isoniazid, ethambutol, linezolid, PA-824 and 8207910 derivatives).

Further provided herein are methods of screening for an agent that reduces drug resistance in Mycobacterium tuberculosis (Mtb). The methods comprise providing an Mtb with a mutant efflux complex and contacting the Mtb with an agent to be tested and a tuberculosis treating agent. Reduced resistance to the tuberculosis treating agent in the presence of the agent to be tested, as compared to a control, indicates the agent to be screened reduces drug resistance in Mtb. A mutant efflux complex can comprise a mutant efflux channel and/or a mutant efflux pump. An efflux channel can, for example, comprise Rv1698 (MctB) or a TolC-like efflux channel. An efflux pump can, for example, comprise Rv0194. An agent to be tested can, for example, be an agent available in a library. The agent to be tested can be an agent that blocks function or expression of the OM protein and can include, for example, small molecules, polypeptides, nucleic acids, or peptidomimetics.

Determining whether reduced resistance to the tuberculosis treating agent occurs in the presence of the agent to be tested involves comparison to a control. A control can include, for example, treating the same mutant Mtb with the tuberculosis treating agent but no agent to be tested. Comparisons of growth characteristics between the two samples determines whether a reduced resistance to the tuberculosis treating agent occurs in the presence of the agent to be tested. If the mutant Mtb treated with the agent to be tested exhibits slower growth characteristics or, alternatively, does not grow at all, as compared to the other mutant Mtb, then the agent to be tested reduces drug resistance in Mtb.

Mtb mutants can be made using methods known in the art. For example, plasmid mediated transposon insertion can be used to create a library of Mtb mutants. See, e.g., Pelicic et al., Proc. Natl. Acad. Sci. USA 94:10955-60 (1997). Using transposon mediated insertional mutagenesis can lead to Mtb mutants that overexpress a protein (e.g., mutations in the promoter that increase expression of the protein), that decreases expression a protein (e.g., mutations in the promoter or start codon that decreases or prevents expression of the protein), that does not affect the function of the protein (e.g., intergenic mutations), or that expresses a mutant form of a protein (e.g., a truncated form of the protein).

There are a variety of sequences that are disclosed on Genbank, at www.pubmed.gov and these sequences and others are herein incorporated by reference in their entireties as are individual subsequences or fragments contained therein. Rv1698 (MctB) copper efflux channel and homologs, variants, mutants, and isoforms thereof are provided herein. For example, the nucleotide and amino acid sequences of Rv1698 (MctB) can be found at GenBank Accession Nos. NC_{—}000962.2 (from nucleotide 191,488 to 192,432) and NP_{—}216214, respectively. Rv0194 efflux pump and homologs, variants, mutants, and isoforms thereof, are also provided herein. The nucleotide and amino acid sequences of Rv0194 can be found at GenBank Accession Nos. NC_{—}000962.2 (from nucleotide 226,877 to 230,461) and NP_{—}214708.1, respectively. Thus provided are the nucleotide sequences of Rv1698 (MctB) and Rv0194 comprising a nucleotide sequence at least about 70%, 75%, 80%, 85%, 90%, 95%, 98%, 99% or more identical to the nucleotide sequence of the aforementioned GenBank Accession Numbers. Also provided are amino acid sequences of Rv1698 (MctB) and Rv0194 comprising an amino acid sequence at least about 70%, 75%, 80%, 85%, 90%, 95%, 98%, 99% or more identical to the sequences of the aforementioned GenBank Accession Numbers. As with all peptides, polypeptides, and proteins, including fragments thereof, it is understood that additional modifications in the amino acid sequence of the Rv1698 (MctB) and Rv0194 polypeptides can be selected to alter the nature or function of the peptides, polypeptides, or proteins.

Nucleic acids that encode the polypeptide sequences, variants, mutants, and fragments thereof are disclosed. These sequences include all degenerate sequences related to a specific protein sequence, i.e., all nucleic acids having a sequence that encodes one particular protein sequence as well as all nucleic acids, including degenerate nucleic acids, encoding the disclosed variants, mutants, and derivatives of the protein sequences. Thus, while each particular nucleic acid sequence may not be written out herein, it is understood that each and every sequence is in fact disclosed and described herein through the disclosed protein sequences.

The polypeptides provided herein have a desired function. Rv1698 (MctB) is a copper efflux channel, responsible for channeling excess copper out of Mycobacterium tuberculosis to achieve a proper homeostasis for the bacteria. Rv0194 is an efflux pump responsible for pumping antibiotics out of Mycobacterium tuberculosis to ensure survival of the bacteria. The polypeptides are tested for their desired activity using the in vitro assays described herein. For example, Mtb mutants used for screening have decreased activity of the efflux complex. The Mtb mutant can have decreased activity of an efflux channel, an efflux pump, or a combination thereof.

The polypeptides described herein can be further modified and varied resulting in maintenance of the desired function, or alternatively, inhibition or dis-inhibition of the desired function. It is understood that one way to define any known modifications and derivatives or those that might arise, of the disclosed genes and proteins herein is through defining the modifications and derivatives in terms of identity to specific known sequences. Specifically disclosed are polypeptides which have at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent identity to Rv1698 (MctB) and Rv0194 and variants provided herein. Those of skill in the art readily understand how to determine the identity of two polypeptides. For example, the identity can be calculated after aligning the two sequences so that the identity is at its highest level.

Another way of calculating identity can be performed by published algorithms. Optimal alignment of sequences for comparison may be conducted by the local identity algorithm of Smith and Waterman, Adv. Appl. Math, 2:482 (1981), by the identity alignment algorithm of Needleman and Wunsch, J. Mol. Biol. 48: 443 (1970), by the search for similarity method of Pearson and Lipman, Proc. Natl. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of identity can be obtained for nucleic acids by, for example, the algorithms disclosed in Zuker, Science 244:48-52 (1989); Jaeger et al., Proc. Natl. Acad. Sci. USA 86:7706-7710 (1989); Jaeger et al., Methods Enzymol. 183:281-306 (1989), which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity and to be disclosed herein.

Protein modifications include amino acid sequence modifications. Modifications in amino acid sequence may arise naturally as allelic variations (e.g., due to genetic polymorphism) or may be produced by human intervention (e.g., by mutagenesis of cloned DNA sequences), such as induced point, deletion, insertion, and substitution mutants. These modifications can result in changes in the amino acid sequence, provide silent mutations, modify a restriction site, or provide other specific mutations. Amino acid sequence modifications typically fall into one or more of three classes: substitutional, insertional, or deletional modifications. Insertions include amino and/or terminal fusions as well as intrasequence insertions of single or multiple amino acid residues. Insertions ordinarily will be smaller insertions than those of amino or carboxyl terminal fusions, for example, on the order of one to four residues unless chimeric polypeptides are desired. Deletions are characterized by the removal of one or more amino acid residues from the protein sequence. Optionally, no more than about from 2 to 6 residues are deleted at any one site within the protein molecule unless entire domains are deleted. Amino acid substitutions are typically of single residues but can occur at a number of different locations at once; insertions usually will be on the order of about from 1 to 10 amino acid residues; and deletions will range about from 1 to 30 residues. Deletions or insertions preferably are made in adjacent pairs, i.e., a deletion of 2 residues or insertion of 2 residues. Substitutions, deletions, insertions or any combination thereof may be combined to arrive at a final construct. Substitutional modifications are those in which at lease one residue has been removed and a different residue inserted in its place. Such substitutions can be made in accordance with the following Table 1 and are referred to as conservative substitutions. Alternatively, when changes in function are desired, nonconservative substitutions can be selected (e.g., proline for glycine).

TABLE 1
Amino Acid Substitutions

| Amino Acid | Substitutions (others are known in the art) |
|---|---|
| Ala | Ser, Gly, Cys |
| Arg | Lys, Gln, Met, Ile |
| Asn | Gln, His, Glu, Asp |
| Asp | Glu, Asn, Gln |
| Cys | Ser, Met, Thr |
| Gln | Asn, Lys, Glu, Asp |
| Glu | Asp, Asn, Gln |
| Gly | Pro, Ala |
| His | Asn, Gln |
| Ile | Leu, Val, Met |
| Leu | Ile, Val, Met |
| Lys | Arg, Gln, Met, Ile |
| Met | Leu, Ile, Val |
| Phe | Met, Leu, Tyr, Trp, His |
| Ser | Thr, Met, Cys |
| Thr | Ser, Met, Val |
| Trp | Tyr, Phe |
| Tyr | Trp, Phe, His |
| Val | Ile, Leu, Met |

Modifications, including the specific amino acid substitutions, are made by known methods. By way of example, modifications are made by site specific mutagenesis of nucleotides in the DNA encoding the protein, thereby producing DNA encoding the modification, and thereafter expressing the DNA in recombinant cell culture. Techniques for making substitution mutations at predetermined sites in DNA having a known sequence are well known, for example M13 primer mutagenesis and PCR mutagenesis.

Provided herein are methods of treating or preventing infection from Mycobacterium tuberculosis (Mtb) in a subject. Such methods include administering an effective amount of an agent that inhibits the activity of an efflux complex. The agent can, for example, comprise a small molecule, a polypeptide, a nucleic acid molecule, a peptidomimetic or a combination thereof. Optionally, the small molecules, polypeptides, nucleic acid molecules, and/or peptidomimetics are contained within a pharmaceutical composition.

Provided herein are compositions containing the provided small molecules, polypeptides, nucleic acid molecules, and/or peptidomimetics and a pharmaceutically acceptable carrier described herein. The herein provided compositions are suitable of administration in vitro or in vivo. By pharmaceutically acceptable carrier is meant a material that is not biologically or otherwise undesirable, i.e., the material is administered to a subject without causing any undesirable biological effects or interacting in a deleterious manner with the other components of the pharmaceutical composition in which it is contained. The carrier is selected to minimize any degradation of the active ingredient and to minimize any adverse side effects in the subject.

Suitable carriers and their formulations are described in Remington: The Science and Practice of Pharmacy, 21st Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally about 5 to about 8 or from about 7 to 7.5. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the agent, e.g., the small molecule, polypeptide, nucleic acid molecule, and/or peptidomimetic, to humans or other subjects.

The compositions are administered in a number of ways depending on whether local or systemic treatment is desired, and on the area to be treated. The compositions are administered via any of several routes of administration, including topically, orally, parenterally, intravenously, intra-articularly, intraperitoneally, intramuscularly, subcutaneously, intracavity, transdermally, intrahepatically, intracranially, nebulization/inhalation, or by installation via bronchoscopy. Optionally, the composition is administered by oral inhalation, nasal inhalation, or intranasal mucosal administration. Administration of the compositions by inhalant can be through the nose or mouth via delivery by spraying or droplet mechanism. For example, in the form of an aerosol.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like.

Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

Compositions for oral administration include powders or granules, suspension or solutions in water or non-aqueous media, capsules, sachets, or tables. Thickeners, flavorings, diluents, emulsifiers, dispersing aids or binders are optionally desirable.

Optionally, the nucleic acid molecule or polypeptide is administered by a vector comprising the nucleic acid molecule or a nucleic acid sequence encoding the polypeptide. There are a number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based deliver systems. Such methods are well known in the art and readily adaptable for use with the compositions and methods described herein.

As used herein, the terms peptide, polypeptide, or protein are used broadly to mean two or more amino acids linked by a peptide bond. Protein, peptide, and polypeptide are also used herein interchangeably to refer to amino acid sequences. It should be recognized that the term polypeptide is not used herein to suggest a particular size or number of amino acids comprising the molecule and that a peptide of the invention can contain up to several amino acid residues or more.

As used throughout, subject can be a vertebrate, more specifically a mammal (e.g. a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient or subject may be used interchangeably and can refer to a subject with a disease or disorder (e.g. tuberculosis). The term patient or subject includes human and veterinary subjects.

A subject at risk of developing a disease or disorder can be predisposed to the disease or disorder, e.g., live, work, or socially come into contact with a subject infected with Mycobacterium tuberculosis. A subject currently with a disease or disorder has one or more than one symptom of the disease or disorder and may have been diagnosed with the disease or disorder.

The methods and agents as described herein are useful for both prophylactic and therapeutic treatment. For prophylactic use, a therapeutically effective amount of the agents described herein are administered to a subject prior to onset (e.g., before infection with Mycobacterium tuberculosis) or during early onset (e.g., upon initial signs and symptoms of tuberculosis). Prophylactic administration can occur for several days to years prior to the manifestation of symptoms of tuberculosis. Prophylactic administration can be used, for example, in the preventative treatment of subjects likely to be exposed to other subjects currently afflicted with tuberculosis. Therapeutic treatment involves administering to a subject a therapeutically effective amount of the agents described herein after diagnosis or development of tuberculosis.

According to the methods taught herein, the subject is administered an effective amount of the agent. The terms effective amount and effective dosage are used interchangeably. The term effective amount is defined as any amount necessary to produce a desired physiologic response. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

As used herein the terms treatment, treat, or treating refers to a method of reducing the effects of a disease or condition or symptom of the disease or condition. Thus in the disclosed method, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refers to an action, for example, administration of a therapeutic agent, that occurs before or at about the same time a subject begins to show one or more symptoms of the disease or disorder, which inhibits or delays onset or exacerbation of one or more symptoms of the disease or disorder. As used herein, references to decreasing, reducing, or inhibiting include a change of 10%, 20%, 30%, 40%, 50%, 60%, 70%, 80%, 90% or greater as compared to a control level. Such terms can include but do not necessarily include complete elimination.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### Example 1

### Rv1698 of Mycobacterium tuberculosis Represents a New Class of Channel-Forming Outer Membrane Proteins

### General Methods

### Bacterial Strains and Growth Conditions

All of the bacterial strains used in this study are listed in Table 2. Mycobacterial strains were grown at 37° C. in Middlebrook 7H9 liquid medium (Difco Laboratories of Becton Dickinson (BD); Franklin Lakes, N.J.) supplemented with 0.2% glycerol, 0.05% Tween 80 or on Middlebrook 7H10 agar (Difco Laboratories) supplemented with 0.2% glycerol unless indicated otherwise. E. coli DH5a was used for all cloning experiments and was routinely grown in Luria-Bertani medium at 37° C. The following antibiotics were used when required at the following concentrations: ampicillin (100 μg/ml for E. coli), kanamycin (30 μg/ml for E. coli; 30 μg/ml for M. smegmatis), and hygromycin (200 μg/ml for E. coli, 50 μg/ml for M. smegmatis).

### Antibiotic Sensitivity Assay

M. smegmatis strains were grown in a 4-ml culture for 2 days at 37° C. to an A₆₀₀ of 0.6-0.8. The cultures were diluted in Middlebrook 7H9 medium to yield ˜5,000 colony-forming units (cfu)/ml. Approximately 500 cfu were streaked out on plates containing the appropriate antibiotic concentrations. As a reference 500 cfu were also plated onto plates without any antibiotic. The number of surviving cells was normalized to the number of cells counted on plates without antibiotic for each strain and expressed as relative colony forming units (% cfu). Colony counts were carried out after 3 days of incubation at 37° C. The concentrations of the antibiotics were: ampicillin, 32 μg/ml; cephaloridin, 3 μg/ml; and chloramphenicol, 6 μg/ml.

### Outer Membrane Permeability for Glucose

The experiments were carried out as previously described (Stahl et al., Mol. Microbiol. 40:451-64 (2001)). To reduce aggregation and clumping, M. smegmatis strains were grown first in 4-ml cultures for 2 days at 37° C. and then filtered through a 5-μm pore size filter (Sartorius; Goettingen, Germany). The filtrates were grown for 2 days at 37° C. and then used to inoculate 100-ml cultures that were grown to an A₆₀₀ of 0.5. The cells were harvested by centrifugation (4° C., 3000 rpm, 10 min), washed once in 2 mM PIPES, pH 6.5, 0.05 mM MgCl₂, and resuspended in the same buffer. The ¹⁴Ct-labeled compounds and their non-labeled analogs were mixed and added to the cell suspensions to obtain a final concentration of 20 μM with 106 cpm. The mixtures were incubated at 37° C., and 1 ml-samples were removed at the indicated times. The cells were filtered through a 0.45-μmpore size filter (Sartorius) and washed with 0.1 M LiCl, and their radioactivity was determined using a liquid scintillation counter. The mean dry weight of the cells in these samples was 1.4±0.4 mg. The uptake rate was expressed as nmol/mg cells.

Construction of Overexpression Vectors for rv1698 for Mycobacteria and E. coli

The gene rv1698 of M. tuberculosis and its homologous gene msmeg₁₃ 3747 of M. smegmatis were amplified by PCR using chromosomal DNA and the oligonucleotide pairs 1698fwd/1698rev and mpoS_SDopt_{—}1/mspTSDrev (Table 2) introducing the restriction sites Pad and Swal. The genes were cloned into pMN016 under the control of the p_{smyc} promoter by using those restriction sites (Stephan et al., Mol. Microbiol. 58:714-30 (2005)) to give the expression vectors pMN035 and pMN451, respectively (Table 2). To purify Rv1698 from mycobacteria, the protein was C-terminally tagged with six histidine residues by PCR using the primer 1698fwd and the 5′-phosphorylated primer mpoTHis (Table 2). The resulting PCR product was digested with Pad and cloned into the backbone of pMN016 digested with Pad and Swal to give pML911. To overexpress and analyze the pore forming activity of recombinant Rv1698, the truncated gene lacking the putative signal sequence of 30 amino acids was fused to a C-terminal His tag by cloning it into the vector pET28+ (Novagen; Gibbstown, N.J.). The gene without its putative leader sequence was amplified from pMN035 by using the oligonucleotides pMS-Seq1 and his rv1698fwd (Table 2). Both, the PCR fragment and pET28b+were digested with restriction endonucleases NdeI and HindIII and ligated to give pML122 (Table 2).

A vector for expression of untagged Rv1698 without the predicted signal peptide in E. coli was constructed using the oligonucleotides mat-rv1698fwd and mat-rvrev introducing the two restriction sites NcoI and NdeI (Table 2). The digested fragment was cloned into the vector pET-16b, which was treated with the same restriction endonucleases to obtain pML141.

TABLE 2
Bacterial strains, plasmids, and oligonucleotides in Example 1. The
annotations Sm^{R}, Gen^{R}, Amp^{R}, Cm^{R}, Hyg^{R} and Kan^{R} indicate resistance to the
antibiotics streptomycin, gentamicin, ampicillin, chloramphenicol, hygromycin and
kanamycin, respectively. The vectors for over-expression in E. coli contain the
rv1698 gene without its predicted leader peptide (marked with the subscript −SP).
Tags of x consecutive histidines for affinity purification are abbreviated as Hisₓ.
Restriction sites in the oligonucleotides used for cloning are underlined.

| Strain | Parent strain and relevant genotype |
|---|---|
| E. coli DH5α | recA1, endA1, gyrA96, thi; relA1,hsdR17(r_{K}⁻,m_{K}⁺), supE44, |
| | φ80ΔlacZΔM15, ΔlacZ(YA-argF)UE169 (Hanahan, J. Mol. Biol. |
| | 166:557-580 (1983)) |
| E. coli | F ompT hsdS_{B}(r_{B}⁻ m_{B}⁻) gal dcm lacYI, pRARE (Cm^{R}) (Studier and |
| Rosetta | Moffatt, J. Mol. Biol. 189:112-30 (1986)) |
| M. smegmatis | M. smegmatis mc²155; Sm^{R} (Sander et al., Mol. Microbiol. 16:991- |
| SMR5 | 1000 (1995)) |
| M. smegmatis | SMR5, ΔmspA::aacC1; Gen^{R}, Sm^{R} (Stahl et al., Mol. Microbiol. 40:451- |
| MN01 | 64 (2001)) |
| M. smegmatis | SMR5, ΔmspA::FRT, ΔmspC::FRT, Sm^{R} (Stephan et al., Mol. Microbiol. |
| ML10 | 58:714-30 (2005)) |
| M. bovis BCG | Pasteur 35739 (ATCC) |
| Plasmid | Parent vector, relevant genotype and properties |
| pMS2 | Co1E1 origin, PAL5000 origin, Hyg^{R} |
| pMN016 | P_{smyc}-mspA, ColE1 origin, PAL5000 origin, Hyg^{R} |
| pMN035 | P_{smyc}rv1698, ColE1 origin, PAL5000 origin, Hyg^{R} |
| pML911 | P_{smyc}, rv1698_{His6}, ColE1 origin, PAL5000 origin, Hyg^{R} |
| pML451 | P_{smyc}, msmeg3754, ColE1 origin, PAL5000 origin, Hyg^{R} |
| pML122 | pET-28b+ derivative, p_{T7}, _{His5}rv1698_{-SP}, Kan^{R} |
| pML141 | pET-16b derivative, p_{T7}, rv1698_{-SP}, Amp^{R} |
| pET-28b+ | pBR322 origin, f1-origin, lacI, Kan^{R} |
| pET-16b | pBR322 origin, lacI, Amp^{R} |
| pMN437 | P_{smyc}, mycgfp2+, ColE1 origin, PAL5000 origin, Hyg^{R} |
| pMV61015.1 | Pₕₛₚ₆₀, rv1818c_{HA}, ColE1 origin, PAL5000, Hyg^{R} |
| Oligonucleotide | Sequence (5' to 3' direction) |
| 1698fwd | GATTACTTAATTAACAGAAAGGAGGTTAATATGATCTCGTTGCGTCAA |
| | CATGCGGTCTCAC (SEQ ID NO: 2) |
| 1698rev | ATATAATTTAAATGGAACACGCCCTAACGCGGGCCTACTG |
| | (SEQ ID NO: 3) |
| mpoS_SDopt_1 | CGTTAATTAAGCAGAAAGGAGGTTAATCTATGATAACGCTACGGGCG |
| | CACGCGATC (SEQ ID NO: 4) |
| mspTSDrev | ATATAATTTAAATGCGCCTCTACTGCGGGACCGTCACCGAAGAC |
| | (SEQ ID NO: 5) |
| mpoTHis | AAATGGACTAGTGGTGGTGGTGGTGGTGCTGGGAAACCGTGACTGAC |
| | ATCGC (SEQ ID NO: 6) |
| pMS-Seq1 | CGTTCTCGGCTCGATGATCC (SEQ ID NO: 7) |
| his_rv1698fwd | ATATACATATGGATACTTTGCTGTCCAGCTTGCGTAG (SEQ ID NO: 8) |
| mat-rv1698fwd | CATTAGCCATGGATACTTTGCTGTCCAGCTTGCGTAG (SEQ ID NO: 9) |
| mat-rvrev | CATTAGCATATGGATAACGTTCTCGGCTCGATGATCC (SEQ ID NO: 10) |
| RT-rv1698fwd | TTGCTGTCCAGCTTGCGTAG (SEQ ID NO: 11) |
| RT-rv1698rev | AGGCGATGCCGAGCAGGT (SEQ ID NO: 12) |

Overexpression, Purification, and Renaturation of Recombinant Rv1698_{His} from E. coli

Cultures of E. coli Rosetta carrying the overexpression vectors pML122 were grown at 37° C. When the culture reached an A₆₀₀ of 1, isopropylthio-β-_{D}-galactosidase was added to a final concentration of 0.5 mM to induce gene expression. After a further 4 hours of incubation, the bacteria were harvested and resuspended in 20 ml of lysis buffer (50 mM Tris-HCl, 100 mM NaCl, 0.5% Triton X-100). The cell suspension was sonicated four times for 20 seconds with 12 watts in 30-s intervals. 0.01 mg/ml DNase and 0.1 mg/ml lysozyme were added and incubated at room temperature for 20 minutes. The broken cells were harvested by centrifugation and resuspended in lysis buffer followed by sonication as described above. This step was repeated twice. Then the broken cells were resuspended in washing buffer (50 mM Tris-HCl, 100 mM NaCl) and sonicated again. Because rRv1698_{His} formed inclusion bodies, the proteins in the pellet were dissolved with 8 M urea, separated from cell debris by centrifugation, and purified using nickel-nitrilotriacetic acid columns (nickel-nitrilotriacetic acid spin kit; Qiagen). The bound proteins were washed and eluted from the column by using a four-step pH gradient with TPU buffer (6 M urea, 0.1 M NaH₂PO₄, 0.01 M Tris) at different pH levels (pH 6.3/5.9/4.8/4.5) according to the recommendations of the manufacturer. The protein fraction at pH 4.5 contained most of the rRv1698_{His} protein. Purified rRv1698_{His} (180 μg/ml) was diluted 100-fold in 25 mM sodium-phosphate buffer (pH 7.5) containing 0.5% n-octylpolyethylene oxide (OPOE) at room temperature to remove urea and renature the protein. The resulting protein was directly used in black lipid bilayer experiments to determine single channel conductance and ion selectivity of the pore as described below. The BCA kit (Pierce; Rockford, Ill.) was used routinely to determine protein concentrations.

Purification of Rv1698_{His} from M. bovis BCG

M. bovis BCG carrying the plasmid pML911 was grown in Middlebrook 7H9 liquid medium supplemented with 10% oleic acid albumin dextrose complex, 0.05% Tween 80, and hygromycin. At an A₆₀₀ of 1, the bacteria were harvested, incubated in a rotatory shaker (200 rpm) with lysozyme 1 mg/ml in phosphate-buffered saline (PBS; 137 mM NaCl, 10 mM potassium phosphate, 2.7 mM KCl, pH 7.4) for 2 hours at 37° C. and disrupted at 4° C. using a Sonicator 3000 ultrasonic liquid processor (Misonix; Farmingdale, N.Y.) in 2 steps of 30 minutes (micro tip, pulsar cycle of 1 second, 9 Watts delivered per cycle). The proteins were solubilized by incubation with 1% SDS in PBS for 18 hours (37° C., 200 rpm). Nonsoluble material was removed by centrifugation (10,000×g, 4° C.) before purifying Rv1698_{His} on nickel-nitrilotriacetic acid-agarose (Qiagen; Valencia, Calif.) using a batch procedure. The bound His-tagged protein was washed and eluted from the resin by using a three-step imidazole gradient (5/20/250 mM) in sodium phosphate buffer (50 mM NaH₂PO₄, pH 7.6, 0.3 M NaCl) according to the recommendations of the manufacturer. The His-tagged protein was eluted with 250 mM imidazole. Because of the high imidazole concentration, the BCA assay was not used, and the purified Rv1698_{His} protein was quantified by using the Bradford protein assay (Bio-Rad; Hercules, Calif.) according to the manufacturer's recommendations. In addition, a calibration curve of band intensities was established with known amounts of bovine serum albumin in Coomassie stained SDS-polyacrylamide gels using image analysis software (LabWorks 4.6, UVP; Upsland, Calif.). Then the amount of Rv1698 was determined relative to the bovine serum albumin reference. Both methods yielded the similar values.

### Lipid Bilayer Experiments

The single channel conductance of Rv1698 protein was analyzed on a custom-made lipid bilayer apparatus as described previously (Heinz and Niederweis, Anal. Biochem. 285:113-120 (2000)). Briefly, the Ag/AgCl electrodes were bathed in a solution of 1 M KCl, 10 mM HEPES, pH 7.0. The lipid membranes were painted from a solution of 1% diphytanoylphosphatidylcholine (DPhPC; Avanti Polar Lipids; Alabaster, Ala.) in n-decane. Before adding the protein, current traces of at least three or more membranes were recorded with the appropriate detergent (0.5% OPOE or 0.1% SDS in 25 mM sodium phosphate, pH 7.5) to exclude any contamination with channel forming activity and to demonstrate that the detergents did not affect the membrane. Then protein was added to both sides of the cuvette. Single channel conductances for more than 100 pores/sample were digitally recorded. The raw data were analyzed using IGOR Pro 5.03 program (WaveMetrics; Colorado Springs, Colo.). These data were further analyzed in SigmaPlot 9.0 (Systat Software; Chicago, Ill.) to generate the figures shown here. The ion selectivity of R1698_{His} was measured as described previously (Niederweis et al., Mol. Microbiol. 33:933-45 (1999)).

Preparation of RNA from M. tuberculosis and RT-PCR Experiments

Total RNA of M. tuberculosis H37Rv was isolated by the Trizol method as recommended by the manufacturer. Briefly, the cultures were grown in 100 ml of 7H9 medium supplemented with 10% oleic acid albumin dextrose complex and 0.05% Tween 80 to log phase. Thirty five ml of the GTC buffer (5 M guanidium thiocyanate, 0.5% sarcosyl, 0.5% Tween 80, 1% β-mercaptoethanol) was added, and the culture was centrifuged at 10,000×g for 10 minutes at 4° C. The pellet was resuspended in 1.5 ml of TRIzol and lysed by agitation with glass beads (FastRNA Tubes-Blue) in a FastPrep® FP120 bead beater apparatus (Bio-101; Qbiogene; Carlsbad, Calif.) for 3×45 seconds at level 6.5. The suspensions were cooled on ice for 5 minutes between agitation steps. 500 μl of chloroform was added, and centrifugation was done for 5 minutes at 14,000×g. The upper phase was transferred to a new tube containing an equal volume of isopropanol. The tubes were incubated for 20 minutes at −80° C. and centrifuged at 14,000×g for 20 minutes at 4° C. The pellet was washed with 70% ethanol, dried, and resuspended in 100 μl of diethylpyrocarbonatetreated water (Ambion; Austin, Tex.). Further purification of samples was performed using Nucleospin→RNAII kit (Macherey-Nagel; Bethlehem, Pa.) following the instructions of the manufacturer. RNA was sonicated to render DNA accessibility to DNase degradation (2×20 seconds at 20% power), 5 minutes on ice between sonications (Stephan et al., BMC Microbiol. 4:45 (2004)). 5-10 μg of sonicated DNA was used for Turbo DNase treatment, which was done according to the manufacturer protocol (Ambion). cDNA synthesis was performed using SuperScript III first strand synthesis system for RT-PCR (Invitrogen; Carlsbad, Calif.) according to the manufacturer protocol using random primers. AccuPrime Pfx SuperMix (Invitrogen) was employed for the PCR. Primers used were RT-rv1698fwd and RT-rv1698rev (Table 2). Thirty five cycles (30 seconds at 95° C., 30 seconds at 58° C., 30 seconds at 68° C.) were run to amplify the cDNA. The PCR products were analyzed using 1% agarose gels, which were stained with ethidium bromide. Primers specific for 16 S rRNA were used as a positive control for RT-PCR.

### Protease Accessibility Assay

Experiments to examine the surface accessibility of Rv1698 were carried out as described previously (Delogu et al., Mol. Microbiol. 52:725-33 (2004)) with minor modifications. M. smegmatis strains carrying the plasmids pMN437, pML911, pML451, and pMV61015.1 were grown in 20 ml of Middlebrook 7H9 medium and harvested as culture reached an A₆₀₀ of about 3.5. The cells were washed once with Tris-buffered saline buffer (0.5 M Tris-HCl, pH 7.2, 150 mM NaCl, 3 mM KCl) and then resuspended in 1 ml of the same buffer. Two aliquots of 150 μl were taken, and proteinase K (Sigma-Aldrich; St. Louis, Mo.) was added to one of the aliquots to a final concentration of 100 μg/ml. After 30 min at 4° C. the reaction was stopped by adding Complete™ EDTA-free protease inhibitor mixture (Roche Applied Science; Indianapolis, Ind.) from a 7-fold stock solution. The samples were immediately centrifuged, washed once in 250 μl of Tris-buffered saline, and resuspended in 75 μl of Tris-buffered saline plus 25 μl of 4× protein loading buffer (160 mM Tris-HCl pH 7.0, 12% SDS, 32% glycerol, 0.4% bromphenol blue). Finally, the samples were boiled for 20 minutes to allow cell lysis and centrifuged again to remove insoluble debris, and 50 μl of the protein extracts were separated on a 10% polyacrylamide gel and analyzed in Western blots using standard protocols (Ausubel et al., Current Protocols in Molecular Biology, John Wiley & Sons, NY (1987)). The program LabWorks 4.6 (UVP) was used for quantitative image analysis to determine the amount of detected Rv1698 protein in treated and untreated samples.

### Analysis of Protein Secondary Structures

Secondary structures of Rv1698 and reference proteins were predicted using the JPred 3 server (Cuff et al., Bioinformatics 14:892-3 (1998); Cuff and Barton, Proteins 34:508-19 (1999)) and the Network Protein Sequence Analysis server. SignalP3.0 (Bendsten et al., J. Mol. Biol. 340:783-95 (2004)) was accessed to examine the presence of signal peptides for Rv1698. The TMHMM2.0 program (Krogh et al., J. Mol. Biol. 305:567-80 (2001); Melen et al., J. Mol. Biol. 327:735-44 (2003)) was used for prediction of hydrophobic transmembrane α-helices. All of the algorithms were used with standard settings unless otherwise noted. The hydrophobicity profile of Rv1698 was calculated using an algorithm that was developed to detect repetitive oscillations of the hydropathy profile (Rauch and Moran, Comput. Methods Programs Biomed. 48:193-200 (1995)) and the Eisenberg hydrophobicity scale of amino acids (Eisenberg et al., J. Cell. Biochem. 31:11-7 (1986)).

### Estimation of the Minimal Size of the Rv1698 Pore

The two-dimensional structures of ampicillin (Compound ID 2174), chloramphenicol (Compound ID 5959), and glucose (Compound ID 5793) were downloaded from PubChemCompound data base at NCBI. The Chem3D Pro 8.0 software (Cambridge-Soft; Cambridge, Mass.) was used to obtain three-dimensional structures of these molecules and to minimize their energy using the MOPAC algorithm. Using the program Chimera (Pettersen et al., J. Comput. Chem. 25:1605-12 (2004)), the molecules were oriented along their longest axis, and the length of the second longest axis was measured between the nuclei of the most distant atoms along this axis. These values were taken as the widths of the molecules and used for a minimal estimate of the pore size of Rv1698.

### Results

The Rv1698 Protein Increases the Susceptibility of an M. smegmatis Porin Mutant to Hydrophilic Antibiotics

A genome-wide secondary structure prediction of all exported proteins identified Rv1698 as a putative outer membrane protein of M. tuberculosis (Song et al., Tuberculosis 88:526-44 (2008)). The genomes of all mycobacteria including M. smegmatis (Msmeg_{—}3747) and Mycobacterium leprae (ML1362) and other closely related bacteria such as Nocardia and Corynebacteria encode a single homolog of Rv1698. This indicated that Rv1698 and its homologs might perform a function specific for the mycolic acid-containing outer membrane of these bacteria.

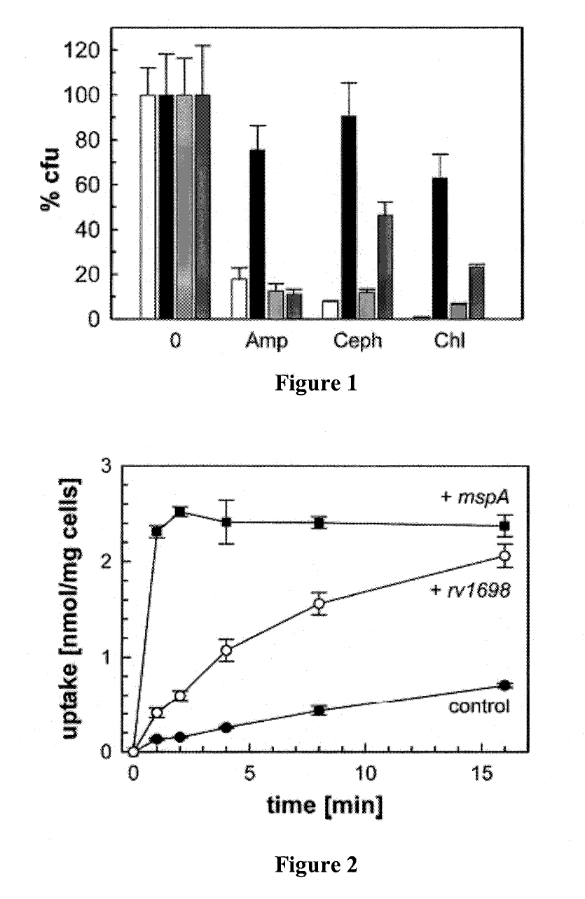

To examine whether this protein might be a porin, the strain M. smegmatis MN01, which lacks the major porin gene mspA, was used. The outer membrane permeability of the ΔmspA mutant to hydrophilic β-lactam antibiotics was decreased 9-fold (Stahl et al., Mol. Microbiol. 40:451-64 (2001)). This resulted in a 16-fold increased resistance toward ampicillin compared with wild-type M. smegmatis (Stephan et al., Antimicrob. Agents Chemother. 48:4163-70 (2004)). Constitutive expression of mspA restored the susceptibility of the ΔmspA mutant MN01 to ampicillin and cephaloridine to wildtype levels as determined by using an agar dilution assay (FIG. 1). The susceptibility of the ΔmspA mutant to these antibiotics was also significantly increased by expression of the rv1698 gene. To exclude that this was an antibiotic-specific effect, whether rv1698 had a similar effect on the efficacy of chloramphenicol against M. smegmatis was examined. Indeed, expression of both mspA and rv1698 significantly increased the susceptibility of the ΔmspA mutant MN01 to chloramphenicol. These results indicated that Rv1698 increased the outer membrane permeability of the M. smegmatis ΔmspA mutant.

Rv1698 Partially Complements the Permeability Defect of Porin Mutants of M. smegmatis

Porin-mediated permeation through the outer membrane is the rate-limiting step for uptake of glucose by M. smegmatis (Stahl et al., Mol. Microbiol. 40:451-64 (2001); Stephan et al., Mol. Microbiol. 58:514-30 (2005)). For example, the minimal permeability coefficient of the ΔmspA ΔmspC mutant ML10 for glucose is 50-fold lower than that of wild-type M. smegmatis (Stephan et al., Mol. Microbiol. 58:514-30 (2005)). Therefore, whether rv1698 could complement the slow uptake of glucose by the porin double mutant ML10 was examined. Glucose at a concentration of 20 μM was taken up by the ML10 strain carrying the empty vector pMS2 with an average rate of 42 μmol/min/mg cells. This rate was increased 5-fold to 224 μmol/min/mg cells in the presence of the rv1698 expression vector pMN035 (Table 2), demonstrating that Rv1698 indeed increased the outer membrane permeability of the porin double mutant ML10 (FIG. 2). However, expression of rv1698 only partially restored the permeability defect of the porin mutant M. smegmatis ML 10 in contrast to the endogenous porin gene mspA (FIG. 2).

### Recombinant Rv1698_{His} is a Channel-Forming Protein

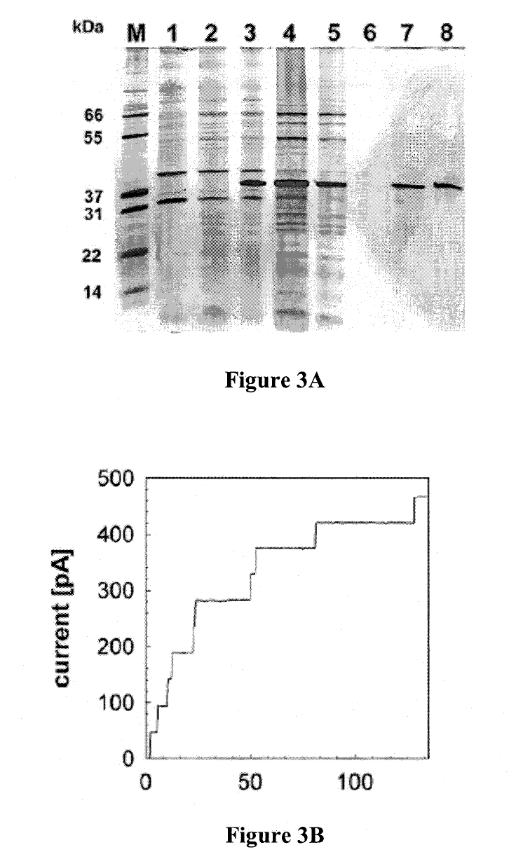

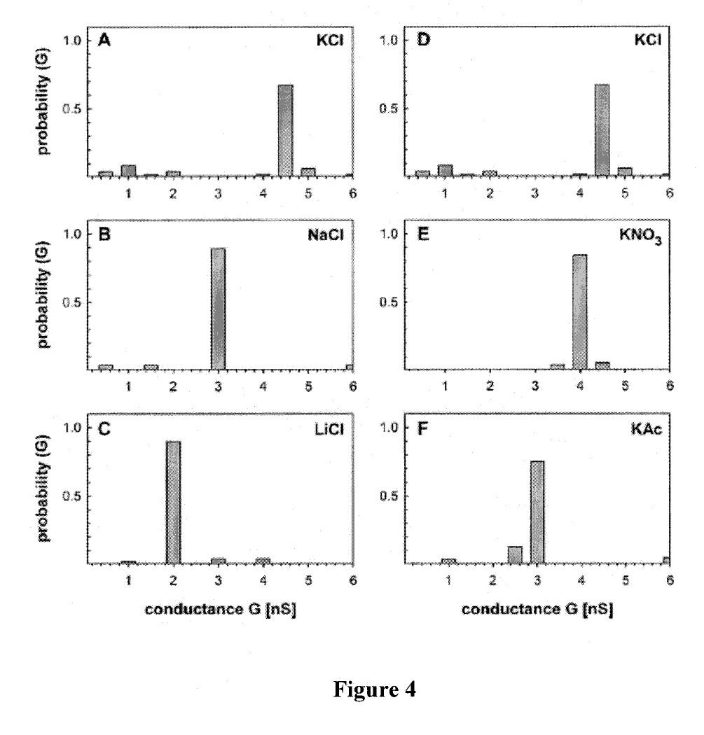

Lipid bilayer experiments provide direct evidence regarding whether a particular protein forms channels within lipid membranes (Niederweis et al., Mol. Microbiol. 33:933-45 (1999)). To this end, a truncated rv1698 gene encoding a protein with an N-terminal histidine tag replacing the predicted signal peptide (amino acids 1-30) was cloned under the control of the T7 promoter in the plasmid pML122 (Table 2). After induction of E. coli Rosetta containing pML122 with isopropylthio-β-_{D}-galactosidase, the bacteria were harvested and disrupted by sonication. Because rRv1698_{His} was insoluble (FIG. 3A, lane 4), the pellet was dissolved with 8 M urea (FIG. 3A, lane 5) and separated from cell debris by centrifugation. The rRv1698_{His} protein was purified by Ni²⁺ affinity chromatography to apparent homogeneity with a yield of 7.4 mg from a 1-liter culture of E. coli as demonstrated by a silver-stained protein gel (FIG. 3A, lane 8). This protein was diluted 1:100 overnight in a 0.5% OPOE-containing buffer to initiate refolding. Nanogram amounts detergent-solubilized rRv1698His protein (3 ng/ml) showed a high channel forming activity after reconstitution in planar lipid membranes (FIG. 3B). No pore was recorded in control experiments when only detergent-containing buffer was added to the lipid bilayer. The single channel conductance of rRv1698_{His} in 1 M KCl was 4.5 nS as determined from 46 reconstitution events in seven membranes (FIG. 4A). Because this channel conductance is almost identical to that of MspA (Niederweis et al., Mol. Microbiol. 33:933-45 (1999)), the activity recorded after addition might have been due to contamination of the bilayer apparatus with minute amounts of highly active and extremely stable MspA (Heinz et al., J. Biol. Chem. 278:8678-85 (2003)). Therefore, purification and renaturation of rRv1698_{His} were repeated. Special care was taken to use fresh buffers and equipment that were not in prior contact with MspA. None of the control measurements with detergent alone showed any pore activity. By contrast, 34 reconstitution events were observed in five membranes after addition of rRv1698_{His} to a final concentration of 1 μg/ml. The single channel conductance of rRv1698_{His} in 1 M KCl was 4.3 nS in excellent agreement with the previous experiments. The reason for the strongly varying channel activity of the recombinant protein is unknown but might be caused by different refolding yields of Rv1698 after dissolving the inclusion bodies.

To exclude that the N-terminal histidine tag modified the channel activity, an rv1698 gene encoding for a rRv1698 protein without the predicted signal peptide and without histidine tag was expressed in E. coli Rosetta using the vector pML141 (Table 2). This truncated rRv1698 protein was exclusively found in inclusion bodies. The single channel conductance of the purified truncated rRv1698 in 1 M KCl was 4.3 nS as determined from 16 reconstitution events in five membranes. Taken together, these experiments showed that (i) recombinant Rv1698 is an integral channel-forming membrane protein, (ii) the predicted signal peptide is not necessary for channel formation in vitro, and (iii) the N-terminal histidine tag does not impair the channel activity of Rv1698. Thus, it is concluded that Rv1698 is a channel-forming protein of M. tuberculosis. It should be noted that reconstitution of the Rv1698 pore in lipid bilayers occurred exclusively in a step-like fashion as shown in FIG. 3B, indicating that the recombinant protein formed open channels upon insertion. These results are consistent with the complementation of the permeability defect of porin mutants of M. smegmatis as shown above. The rapid reconstitution within lipid bilayers also demonstrates that recombinant Rv1698 is an integral membrane protein.

### Ion Selectivity of the Rv1698 Channel

To further characterize the pore formed by Rv1698 and to identify properties that might distinguish the Rv1698 and MspA channels, the ion selectivity of purified rRv1698_{His} was determined by lipid bilayer experiments. To this end, single channel conductance experiments were done in the presence of 1 M solutions of chloride salts with different cations and potassium salts with different anions. FIG. 4 shows that the single channel conductance of purified rRv1698_{His} was influenced considerably by the salt composition. The channel conductivity of rRv1698_{His} decreased significantly with the increasing radius of the hydrated cation in chloride salts (FIG. 4 and Table 3). For example, the single channel conductance of rRv1698_{His} in 1 M LiCl was less than half of that in 1 M KCl. A similar effect was observed with increasing radii of the hydrated anions in potassium salts (FIG. 4 and Table 3). Thus, it is concluded that cations and anions move with a similar rate through the rRv1698_{His} pore. The conductance of the Rv1698 pore linearly decreased with increasing size of the hydrated cations in the same manner as their specific conductance decreased in water. Thus, rRv1698_{His} forms a wide water-filled channel. It should be noted that the channel activity was extremely low in experiments with RbCl as the electrolyte so that only a few pores were recorded (Table 3). Without meaning to be limited by theory, this may indicate that the integration of open channels was inhibited by RbCl either by preventing insertions into the membrane or by blocking the channel.

Another interesting result was the significant reduction of the single channel conductance in 1 M Tris-HCl at pH 6 compared with pH 8 (Table 3). This may either indicate pH gating of the Rv1698 channel or a proton-induced conformational change of the constriction zone that causes a decreased conductivity for Tris-HCl. Both mechanisms were observed for porins of Gram-negative bacteria (Liu and Delcour, FEBS Lett. 434:160-4 (1998); varma et al., Biophys. J. 90:112-23 (2006)).

To quantify the ion selectivity of rRv1698_{His}, zero current potentials of membranes containing several hundred rRv1698 pores were measured in the presence of salt gradients. A 3-fold KCl gradient resulted in a potential of 16.0±0.15 mV, which was positive at the more dilute side. Using the Goldman-Hodgkin-Katz equation (Benz et al., Biochim. Biophys. Acta 551:238-47 (1979)), this is equivalent to a permeability ratio of cations over anions Pc/Pa of 2.5±1.6. This weak preference of cations over anions is consistent with the results of the single channel experiments in different electrolytes. It is concluded that the rRv1698_{His} channel has little charge preference in contrast to the marked cation preference of the MspA pore, which shows a permeability ratio of cations over anions Pc/Pa of 6.6±0.3 (Niederweis et al., Mol. Microbiol. 33:933-45 (1999)). These results also demonstrated that the channel activity observed with purified rRv1698_{His} is indeed a genuine activity of the rRv1698_{His} protein and does not result from contamination with the extremely stable MspA pores.

TABLE 3
Single channel conductances of purified recombinant Rv1698 in
different electrolytes. The lipid bilayer experiments were done in
the presence of 1M of the respective electrolyte. Protein was
added on both sides of the DPhPC membrane starting at 6 ng/ml,
and its concentration was increased in steps of 6 ng/ml until pores
were detected. The salt solutions were buffered in 10 mM MES at
pH 6 or as indicated above. The ion radii were taken from Trias and
Benz, Mol. Microbiol. 14: 283-290 (1994) and Tansel et al., Separ.
Purif. Technol. 51: 40-7 (2006). No reference was found for the radius
of the hydrated acetate anion (NF).

| | Cation | Anion | Most Freq. | Avg. | | |
|---|---|---|---|---|---|---|
| | Rad. | Rad. | conduct. | conduct. | No. | No. |
| Salt | (nm) | (nm) | (nS) | (nS) | steps | memb. |
| Tris-Hcl, | 0.321 | 0.195 | 0.5 | 0.51 | 52 | 6 |
| pH 6 | | | | | | |
| Tris-HCl, | 0.321 | 0.195 | 1 | 0.95 | 50 | 6 |
| pH 8 | | | | | | |
| LiCl | 0.216 | 0.195 | 2 | 2.1 | 48 | 5 |
| NaCl | 0.163 | 0.195 | 3 | 2.95 | 27 | 5 |
| Kac | 0.110 | NF | 3 | 3.04 | 80 | 5 |
| KNO3 | 0.110 | 0.340 | 4 | 4.15 | 70 | 6 |
| KCl | 0.110 | 0.195 | 4.5 | 4.1 | 46 | 7 |
| NH4Cl | 0.110 | 0.195 | 4.5 | 4.94 | 81 | 6 |
| RbCl | 0.105 | 0.195 | — | 4.75 | 8 | 6 |
| CsCl | 0.106 | 0.195 | 4 | 4.41 | 108 | 5 |

The rv1698 Gene and its Homologs are Expressed in Mycobacteria

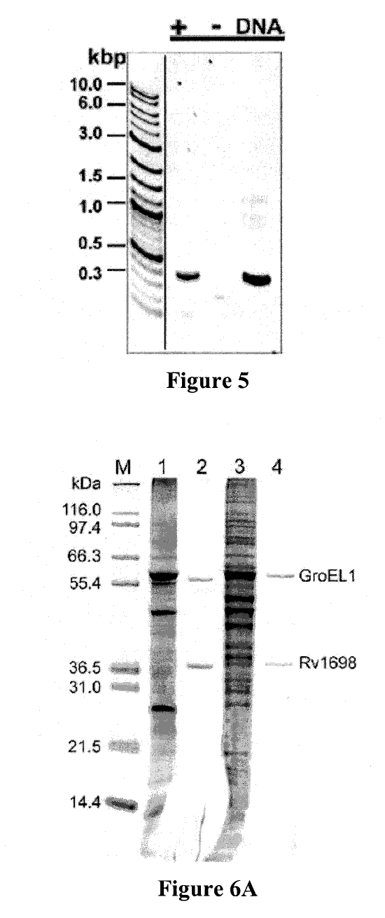

Reverse transcription PCR(RT-PCR) was employed to examine whether rv1698 is expressed in M. tuberculosis grown under standard conditions. Total mRNA was purified from wild-type M. tuberculosis H37Rv. As shown in FIG. 5, PCR yielded a 400-bp DNA fragment when the RNA sample was incubated with reverse transcriptase (lane +). This product was identical in length to a PCR product obtained from chromosomal DNA (FIG. 5, lane DNA) and to the theoretical length of the amplified region of the rv1698 gene, indicating that the PCR was specific. By contrast, no PCR fragment was obtained when reverse transcriptase was omitted, demonstrating that the prepared RNA was not contaminated with DNA (FIG. 5, lane −). These results show that the rv1698 gene is transcribed in M. tuberculosis grown under standard growth conditions. Similar results were obtained for M. bovis BCG and M. smegmatis. Thus, it is concluded that rv1698 is expressed in all three mycobacteria. These findings are supported by the detection of 36-kDa bands in Western blot experiments with SDS extracts of M. tuberculosis, M. bovis BCG, and M. smegmatis using an Rv1698-specific antiserum.

Channel Forming Activity of Rv1698His Purified from M. bovis BCG

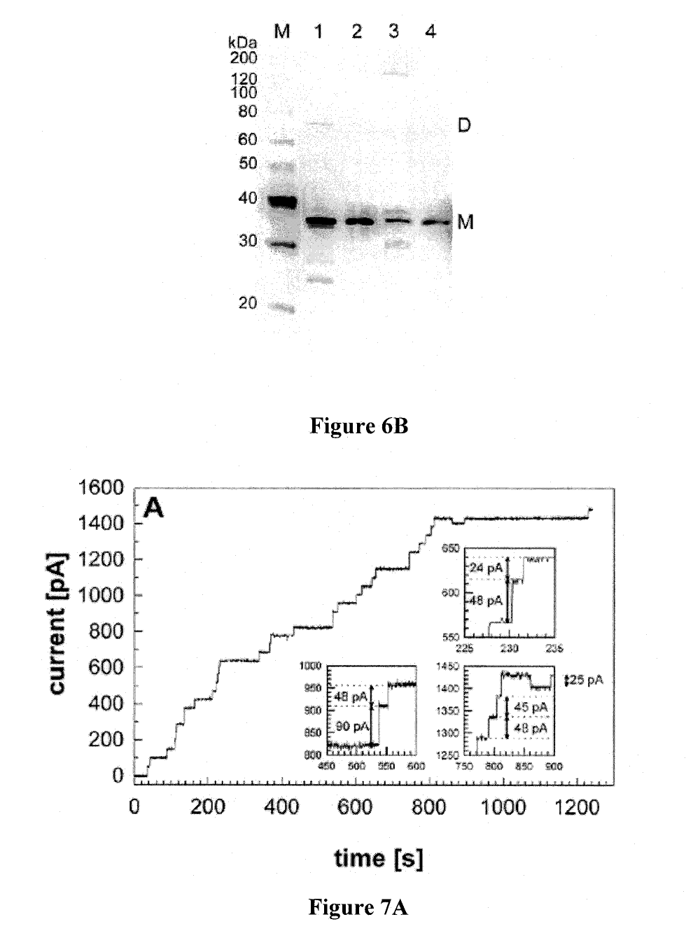

To examine whether the native Rv1698 protein has the same channel activity as the recombinant protein purified from E. coli and to exclude that the channel activity originated from folding artifacts or contaminations from E. coli porins, Rv1698 was purified from M. bovis BCG. To this end, the rv1698 gene encoding a C-terminal histidine tag was expressed from the plasmid pML911 (Table 2) in M. bovis BCG. The proteins were extracted from cell lysates by 1% SDS, and the SDS extract was purified by nickel affinity chromatography (FIG. 6A). Mass spectroscopy of tryptic fragments revealed that the protein with an apparent molecular mass of 36 kDa is indeed Rv1698. Mass spectroscopy also identified the protein with an apparent molecular mass of 57 kDa as GroEL1 (BCG3487c). GroEL1 is a cytoplasmic chaperone that possesses a naturally histidine-rich C-terminal region in mycobacteria (Ojha et al., Cell 123:861-73 (2005)). Analysis of these samples for reactivity with an Rv1698-specific antiserum in Western blots showed that similar amounts of recombinant Rv1698 were produced from plasmid pML911 in M. bovis BCG and M. smegmatis (FIG. 6B). Further, small amounts of apparently dimeric Rv1698 were detected in the sample purified from M. bovis BCG (FIG. 6B). This indicated that Rv1698 might be an oligomeric protein.

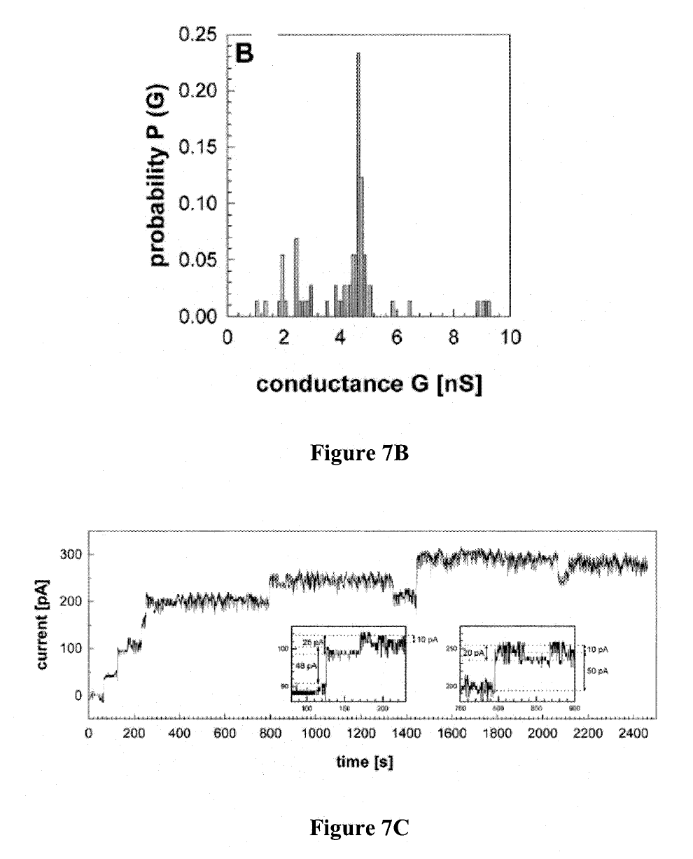

Lipid bilayer experiments were done to examine whether Rv1698 protein isolated from M. bovis BCG also has channel forming activity. No insertion events were detected when buffer was added to the DPhPC membrane. When 300 ng of purified Rv1698His was added to the same membrane, a rapid stepwise current increase was observed (FIG. 7A). More than 100 pores were recorded in seven membranes. The most frequent reconstitution events had conductances of 4.5 to 4.8 nS (FIG. 7B). These results are very similar to the conductance of 4.5 nS determined for the recombinant Rv1698His protein purified from E. coli.

A significant number of Rv1698_{His} channels with smaller conductances of ˜2-2.5 nS and 1.0-1.5 nS were also recorded (FIGS. 7A and 7C). Subconductance states are frequently observed for oligomeric porins of E. coli and other Gram-negative bacteria (Basle et al., Biochim. Biophys. Acta 1664:100-7 (2004)), indicating that Rv1698 might indeed form oligomers. It should be noted that lipid bilayer experiments are not really suitable to quantify the activity of channel proteins because different membranes have to be used in each experiment. This can drastically alter the reconstitution frequency of proteins. In conclusion, purified Rv1698 protein expressed in M. bovis BCG and E. coli showed identical single channel activities, demonstrating unequivocally that Rv1698 is a pore protein.

### Rv1698 is a Surface-Accessible Protein

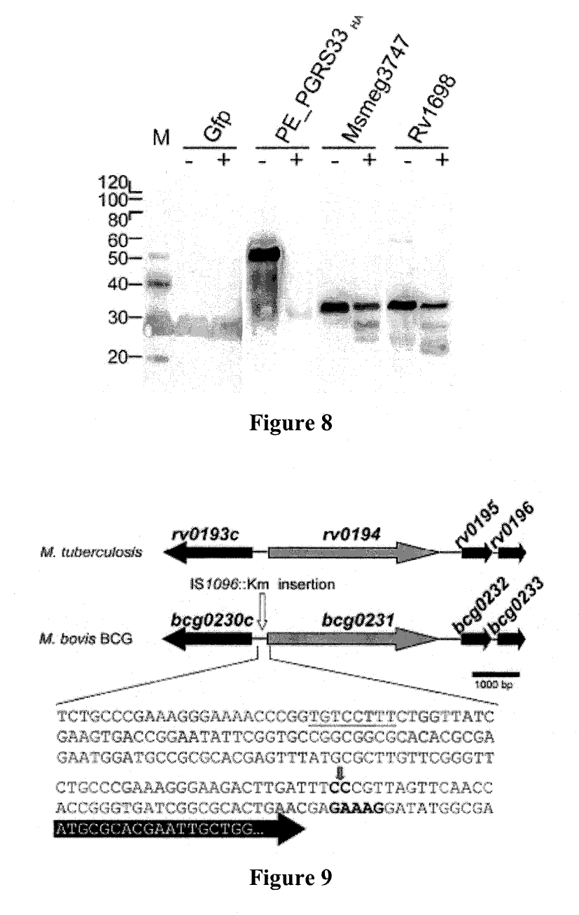

Insertion of large, open, water-filled channel proteins such as porins (Guilvout et al., EMBO J. 25:5241-9 (2006)) or colicins (Cascales et al., Microbiol. Mol. Biol. Rev. 71:158-229 (2007)) into the inner membrane of bacteria is a lethal event, most likely because of the immediate breakdown of the proton gradient. Considering its channel characteristics, it was believed that Rv1698 is an outer membrane protein. To determine the subcellular localization of Rv1698 and to examine whether the Rv1698 protein has surface-exposed loops, protease accessibility was employed as previously described for the surface protein PE_PGRS33 encoded by the rv1818c gene of M. tuberculosis (Delogu et al., Mol. Microbiol. 52:725-33 (2004)). Proteinase K cleaves Msmeg_{—}3747 in 160 and Rv1698 in 158 positions evenly distributed along the entire protein molecule. Thus, in principle, even small surface-exposed loops should be cleaved if Rv1698 is accessible to the protease in whole cells. Green fluorescent protein and PE_PGRS33HA were used as controls for a cytoplasmic protein and as a surface-exposed protein (Delogu et al., Mol. Microbiol. 52:725-33 (2004)), respectively. The signal for green fluorescent protein is identical in both samples, indicating that the cell envelope was intact during proteinase K treatment (FIG. 8). By contrast, the PE_PGRS33HA protein disappeared, demonstrating that PE_PGRS33HA is surface-accessible consistent with previous results (Delogu et al., Mol. Microbiol. 52:725-33 (2004); Cascioferro et al., Mol. Microbiol. 66:1536-47 (2007)). Importantly, the intensities of the bands of the full-length Msmeg_{—}3747 and Rv1698 was reduced by 60% upon proteinase K treatment, demonstrating that both proteins are surface-exposed. It should be noted that the detection of smaller fragments of Msmeg_{—}3747 and Rv1698 was only possible because of the use of an Rv1698-specific antiserum. This is in contrast to the reference protein PE_PGRS33HA, which disappeared completely most likely because of the removal of the hemagglutinin tag from the protein by proteinase K (FIG. 8). Further, the observation of shorter peptides also indicated that some parts of Msmeg_{—}3747 and Rv1698 were protected from proteinase K cleavage, probably because of domains buried in the outer membrane.

### Example 2

### Identification of Novel Multidrug Efflux Pump of Mycobacterium tuberculosis

### General Methods

### Chemicals and Enzymes.

Hygromycin B was purchased from Calbiochem (Gibbstown, N.J.). All other chemicals were purchased from Merck (Whitehouse Station, N.J.), Roth (Watertown, N.Y.), or Sigma (St. Louis, Mo.) at the highest purity available. Enzymes for DNA restriction and modification were from New England Biolabs (Ipswich, Mass.) and Invitrogen (Carlsbad, Calif.). Oligonucleotides were obtained from IDT (Coralville, Iowa).

### Bacterial Strains, Media, and Growth Conditions.

Escherichia coli DH5a was used for cloning experiments and was routinely grown in Luria-Bertani broth at 37° C. Mycobacterium smegmatis strains were grown at 37° C. in Middlebrook 7H9 medium (Difco Laboratories of Becton Dickinson (BD); Franklin Lakes, N.J.) supplemented with 0.2% glycerol and 0.05% Tween 80 or on Middlebrook 7H10 plates supplemented with 0.5% glycerol. M. bovis BCG was grown in Middlebrook 7H9 medium (Difco) supplemented with 0.2% glycerol, 0.05% Tween 80, and 10% oleic acid-albumindextrose-catalase (OADC; Remel) or on Middlebrook 7H10 plates supplemented with 0.5% glycerol and 10% OADC (Remel). Antibiotics were used when required at the following concentrations: hygromycin, 200 μg/ml for E. coli and 50 μg/ml for mycobacteria; kanamycin, 30 μg/ml.

### Construction of Plasmids.

Previous expression vectors were based on transcriptional fusions in which the Shine-Dalgarno sequence had to be included in the forward primer (Kaps et al., Gene 278:115-24 (2001)). To provide an alternative cloning strategy with much shorter forward primers, a Pad restriction site which is not present in the M. tuberculosis genome was used, making cloning with this enzyme very convenient, and was engineered between the gene and the Shine-Dalgarno sequence in this vector backbone. To this end, the mspA gene was amplified by PCR using pMN006 (Stahl et al., Mol. Microbiol. 40:451-64 (2001)) as a template with the oligonucleotides pMS-Seq1 and MspA_SD (Table 4) that introduced SphI and Pad restriction sites and a synthetic Shine-Dalgarno sequence which efficiently initiates translation of gfp. The PCR fragment was digested with SphI and HindIII and cloned into the plasmid pMN013 (Kaps et al., Gene 278:115-24 (2001)) digested with the same restriction endonucleases. This cloning step yielded the vector pML653 in which the Shine-Dalgarno sequence was separated from the translation start site by a Pad restriction site. In this expression vector, genes can be cloned as translational fusions using the restriction sites PacI/HindIII. Promoters can be exchanged using the SpeI and SphI sites.

Then, the rv0194 gene was amplified from genomic DNA of M. tuberculosis H37Rv using the oligonucleotides rv0194 F4 and rv0194_Hind2, which introduced the HindIII restriction site at the 3′-end (Table 4). The rv0194 PCR fragment was digested with HindIII and cloned into pML653 digested with Pad. The 5′-overhanging ends of the Pad sites were removed by T4 DNA polymerase following HindIII restriction digestion to obtain a 5-bp distance between the Shine-Dalgarno sequence and the rv0194 translation start site in the overexpression vector pML655. In addition the rv0194 expression cassette, pML655, features the pAL5000 origin for replication in mycobacteria, the ColE1 origin for replication in E. coli, and a hyg resistance gene.

TABLE 4
Oligonucleotides used. Restriction sites are underlined. The sequence
shown in italics is the Shine-Delgarno sequence of the mspA gene.
Recognition site for T7 RNA polymerase is shown in bold.

| Oligonucleotide | Sequence (5'-3') |
|---|---|
| pMS-Seq1 | CGTTCTCGGCTCGATGATCC (SEQ ID NO: 7) |
| MspA_SD | CGGCATGCAGAAAGGAGGTTAATTAATGAAGGCAATCAGTCGGGT |
| | (SEQ ID NO: 13) |
| Rv0194_F4 | TAATGCGCACGAATTGCTGGTGG (SEQ ID NO: 14) |
| Rv0194_Hind2 | GCAAGCTTGTCAACTCGCCACCCATTCG (SEQ ID NO: 15) |
| Sa1gd | TAGCTTATTCCTCAAGGCACGAGC (SEQ ID NO: 16) |
| IS2 | GAGGCGGCAGAAAGTCGTCAGGTCAG (SEQ ID NO: 17) |
| Tn-mut_seq2 | CAACGTGCGAGTCACGCTGTC (SEQ ID NO: 18) |
| Tn_mut_seq4 | CTTCTGCAGCAACGCCAGGTCCACACTG (SEQ ID NO: 19) |
| Rv0194_F1 | GGCAAATCCACGTTGGCGTC (SEQ ID NO: 20) |
| Rv0194_rev_T7 | CTAATACGACTCACTATAGGGAGACGGCAGAGGTCGGGTCGTCC |
| | (SEQ ID NO: 21) |
| 16SNbfw | TGCTACAATGGCCGGTACAAA (SEQ ID NO: 22) |
| 16SrevT7Prom | CTAATACGACTCACTATAGGGAGACGCTTCCGGTACGGCTACCT |
| | (SEQ ID NO: 23) |
| tnpA_rev | CGAAGGTCAGCGGGTGCTCA (SEQ ID NO: 24) |
| Aph2 | CTCACCGAGGCAGTTCCATA (SEQ ID NO: 25) |

Construction and Analysis of a Transposon Library of M. bovis BCG.

The suicide plasmid vector pPR32, containing IS1096::Km, was used to generate a transposon insertion mutant library of M. bovis BCG as described previously (Pelicic et al., Proc. Natl. Acad. Sci. USA 94:10955-60 (1997)). The vector pPR32 was electroporated into M. bovis BCG. After recovery at 32° C., the bacteria were plated on 7H10 agar containing kanamycin and incubated at 32° C. for 5 to 7 weeks. The colonies were streaked on plates containing kanamycin and gentamicin to prevent the premature loss of the plasmid. Clones were picked from five Kmr/Gmr candidates and transferred into 7H9 liquid medium supplemented with kanamycin and gentamicin. Incubation was at 32° C. for 3 to 4 weeks. The cultures were filtered through a filter with a pore size of 5-μm (Sartorius; Goettingen, Germany) to remove cell clumps. The filtrate was grown for two further weeks until an optical density at 600 nm (OD₆₀₀) of 0.4 to 0.5 was achieved. Approximately 107 cells were plated on 7H10 agar containing kanamycin and 2% sucrose. Incubation of the plates occurred at 39° C. for 3 weeks. Counterselection against the vector pPR32 and selection with kanamycin resulted in approximately 7,500 transposon mutants. This corresponds to a transposon efficiency of 1.5×10⁻³. Ten colonies were arbitrarily picked, and chromosomal DNA was prepared and analyzed by Southern blotting as described elsewhere (Stahl et al., Mol. Microbiol. 40:451-64 (2001)) to examine the randomness of the IS1096::Km insertions. To select for ampicillin-resistant mutants, the library was washed from the plates, passed through a 5-μm filter, and plated on 7H10 plates supplemented with 100 μg/ml of ampicillin. To identify the insertion sites of the transposon, ligation-mediated PCR was employed as described previously (Prod'hom et al., FEMS Microbiol. Lett. 158:75-81 (1998)). Chromosomal DNA was prepared and used as a template for PCR with the primers Salgd and Tn_mut_seq2 or Tn_mut_seq4 and IS2; primers Salgd and IS2 were used for sequencing (Table 4). The resulting sequences were compared with the M. bovis BCG genome sequence using Blast analysis (http://genolist.pasteur.fr/BCGList).

### RNA Preparation.

Total RNA of M. bovis BCG was isolated by the Trizol method as recommended by the manufacturer (Invitrogen). Briefly, cultures were grown in 30 to 60 ml of corresponding medium until late log phase. A 35-ml volume of GTC buffer (5 M guanidium thiocyanate, 0.5% sarcosyl, 0.5% Tween 80, 1% β-mercaptoethanol) was added and centrifuged at 10,000×g for 10 minutes at 4° C. The pellet was resuspended in 1.5 ml Trizol and lysed by agitation with glass beads (FastRNA Tubes-Blue) in a FastPrep FP120 bead beater apparatus (Bio-101) three times for 45 seconds at level 6.5. Suspensions were cooled on ice for 5 minutes between agitation steps. A 500-μl volume of chloroform was added, and centrifugation was done for 5 minutes at 14,000×g. The upper phase was transferred to a new tube containing an equal volume of isopropanol. Tubes were incubated for 20 minutes at −80° C. and centrifuged at 14,000×g for 20 minutes at 4° C. The pellet was washed with 70% ethanol, dried, and resuspended in 100 μl distilled water. Further purification of samples was performed using a Nucleospin→RNAII kit (Macherey-Nagel; Bethlehem, Pa.) following the instructions of the manufacturer.

### Dot Blot Analysis.

The probe for the rv0194 gene was amplified from pML655 by PCR using the primers Rv0194_F1 and Rv0194_rev_T7 (Table 4). The probe for the 16S rRNA gene was amplified from chromosomal DNA of M. bovis BCG using the primers 16SNbfw and 16SrevT7Prom (Table 4). A recognition site for T7 RNA polymerase was added to the 5′-ends of the reverse primers (Table 4). The probes were labeled with digoxigenin by in vitro transcription. The dot blot experiments were carried out as described previously (Hillman et al., J. Bacteriol. 189:958-67 (2007)). The amount of RNA was quantified photometrically. A 7.2-μg aliquot of RNA was spotted in triplicate onto the blot for each sample. To obtain a visible signal for the bcg0231 mRNA in comparison to the standard 16S rRNA, the exposure time of the blot was increased to 700 s. The LabWorks 4.6 software (UVP; Upsland, Calif.) was used for image analysis of the dot blot. The lane profile of the dots was analyzed to examine saturation of the signals. The amount of RNA in the dots was quantified using integrated optical density analysis. The signals for the bcg0231 transcripts were normalized to those of 16S rRNA in the same sample.

### Determination of Antibiotic Susceptibility.

To determine MICs of M. smegmatis and M. bovis BCG strains, a microplate Alamar blue assay (MABA) was used as described previously (Franzblau et al., J. Clin. Microbiol. 36:362-6 (1998)) with some modifications. Final drug concentrations for M. smegmatis were as follows: ampicillin, 62.5 to 2,000 μg/ml; erythromycin and vancomycin, 0.3125 to 10 μg/ml; chloramphenicol and novobiocin, 2 to 64 μg/ml; tetracycline, 0.01875 to 0.6 μg/ml; kanamycin, 0.156 to 5 μg/ml; ciprofloxacin, ofloxacin, and levofloxacin, 0.8 to 25.6 μg/ml. Final drug concentrations for M. bovis BCG were as follows: ampicillin, 62.5 to 2,000 μg/ml; vancomycin, 1.25 to 40 μg/ml; streptomycin, 0.25 to 8 μg/ml; chloramphenicol, 4 to 128 μg/ml; tetracycline, 0.5 to 16 μg/ml. The MICs were defined as the lowest concentration of antibiotic which reduced the viability of the culture by at least 90% as determined by fluorescence measurements at room temperature in top-reading mode at an excitation wavelength of 530 nm and an emission wavelength of 590 nm using a Synergy HT reader (Bio-Tek; Winooski, Vt.).

### β-Lactamase Activity Assay.

The β-lactamase activity of M. bovis BCG was determined by measuring the hydrolysis of nitrocefin by whole cells as described elsewhere (Danilchanka et al., Antimicrob. Agents Chemother. 52:3127-34 (2008)). Briefly, cells of M. bovis BCG strains were grown to saturation (OD₆₀₀, 2.0 to 4.0). Culture supernatants were filtered through 0.2-μm filters (Pall Corporation; East Hills, N.Y.) twice to obtain cell-free culture filtrates. To obtain lysates, cells were pelleted and washed in ice-cold 1× phosphate-buffered saline (PBS) buffer (pH 7.4). The cell pellets were resuspended in a 1/30 volume of 1×PBS containing corresponding amounts of protease inhibitor cocktail (Sigma) and DNase I (New England Biolabs). Cells were disrupted by agitation with glass beads (FastRNA Tubes-Blue) in a FastPrep FP120 bead beater apparatus (Bio-101) twice for 30 seconds at level 6.0 with 5 minutes of rest on ice between agitations. Cell debris was removed by centrifugation and filtered twice through 0.2-μm filters. Protein concentrations were determined using a bicinchoninnic acid protein assay kit (Pierce; Rockford, Ill.). Nitrocefin was added to a final concentration of 200 μM in 1×PBS (pH 7.4), and hydrolysis was monitored as a change in absorbance at 490 nm using a microplate reader (Synergy HT; Bio-Tek). The activities of β-lactamases for each strain were determined as the A₄₉₀ min⁻¹ mg of total protein⁻¹.

### Accumulation of Ethidium Bromide by Mycobacteria.

The accumulation of ethidium bromide by mycobacteria was measured as described previously with some modifications (Margolles et al., Biochemistry 38:16298-306 (1999)). M. smegmatis was grown to early exponential phase (OD₆₀₀, 0.6 to 1.0). The cells were pelleted by centrifugation at room temperature, resuspended in uptake buffer (50 mM KH2PO4 [pH 7.0], 5 mM MgSO4), diluted to an OD₆₀₀ of 0.5, and preenergized with 25 mM glucose for 5 minutes. One hundred microliters of cells was added per well of black, clear-bottomed 96-well microplates (Greiner Bio-One; Monroe, N.C.). Ethidium bromide was added to a final concentration of 20 μM, and its entry was measured at room temperature in top-reading mode at an excitation wavelength of 530 nm and an emission wavelength of 590 nm using a Synergy HT reader (Bio-Tek). When required, reserpine was added after 8 minutes of incubation with ethidium bromide at a final concentration of 0.1 mM.

Susceptibility of M. smegmatis to Ethidium Bromide.

The susceptibility of M. smegmatis to ethidium bromide was tested as described previously (Farrow and Rubin, J. Bacteriol. 190:1783-91 (2008)). Briefly, M. smegmatis was grown overnight in Middlebrook 7H9 medium supplemented with 0.05% Tween 80 and 50 μg/ml hygromycin and filtered through a 5-μm filter (Sartorius) to remove cell clumps. Cells were diluted in the same medium to an approximate OD₆₀₀ of 0.04. Bacterial growth was monitored by measuring the optical density of the cultures at 600 nm. Ethidium bromide was added to the cultures at final concentrations from 1.56 μM to 12.5 μM. When required reserpine was added at a final concentration of 8 mM.

### Results

Isolation of M. bovis BCG Mutants Resistant to Ampicillin.

To identify molecular mechanisms of resistance of slowly growing mycobacteria such as M. tuberculosis to β-lactam antibiotics, M. bovis BCG was used as a model organism and generated a transposon library. The transposon IS1096::Km was chosen, which inserts randomly with a high frequency into mycobacterial genomes (McAdam et al., Infect. Immun. 63:1004-12 (1995)). A culture of a clone from M. bovis BCG containing pPR32 with the transposon IS1096::Km (Pelicic et al., Proc. Natl. Acad. Sci. USA 94:10955-60 (1997)) was plated under conditions nonpermissive for replication of the vector, and this yielded approximately 7,500 kanamycin-resistant clones. To examine the uniqueness of the insertions, chromosomal DNA was prepared from 10 randomly selected clones from the library. Southern blot analysis showed the presence of the transposon in all clones at different positions in the chromosome. This indicated a random transposition of IS1096::Km into the chromosome of M. bovis BCG. To select mutants with a high resistance to β-lactam antibiotics, the library was washed from the plates and filtered to remove cell clumps. Serial dilutions were plated on 7H10 agar with 100 μg/ml ampicillin, on which wild-type (wt) M. bovis BCG did not grow. Seventy-eight ampicillin-resistant mutants were obtained. MICs of ampicillin for all transposon mutants were higher than 62.5 μg/ml for wt M. bovis BCG as determined in a MABA. Twenty-one mutants were completely resistant to ampicillin (≧2,000 μg/ml), while 11 mutants showed a moderate resistance with MICs of 250 to 500 μg/ml (Table 5). Twenty mutants with MICs lower than 250 μg/ml were excluded from further analysis.

TABLE 5
Bioinformatic analysis of insertion sites of the mutants resistant to ampicillin.

| | M. bovis | M. tuber- | | MIC of | |
|---|---|---|---|---|---|
| Group | BCG | culosis | Pos. (bp) | amp. | |
| and Strain | gene | gene | of insert. | (mg/ml) | Gene function |
| Group A | | | | | |
| ML1075 | gca | rv0112 | +899 | 2000 | Putative GDP-mannose 4,6-dehydratase; |
| | | | | | LAM synthesis |
| ML1010 | ppe12 | rv0755c | +43 | 500 | PPE family protein |
| ML1041 | ppe24 | rv1753c | +1707 | >2000 | PPE family protein |
| ML1061 | lppA | rv2543 | +19 | >2000 | Putative lipoprotein |
| ML1058 | lprR | rv2203c | +320 | 2000 | Putative lipoprotein |
| ML1064 | lppB | rv2544 | +569 | 2000 | Putative lipoprotien |
| ML1036 | agpS | rv3107c | +277 | 250 | Putative alkylhydroxyacetone-phosphate |
| | | | | | synthase |
| ML1007 | ppe53 | rv3159c | +988 | 2000 | PPE family protein |
| ML1040 | papA2 | rv3820c | +1390 | 2000 | Putative polyketide synthesis-assoc. protein; |
| | | | | | sulfolipid synthesis |
| ML1047 | fadD23 | rv3826 | +544 | 2000 | Putative fatty acid-coenzymeA ligase; |
| | | | | | sulfolipid synthesis |
| Group B | | | | | |
| ML1069 | gmhA | rv0113 | +205 | 2000 | Putative phosphoheptose isomerase |
| ML1013 | cpsY | rv0806c | +923 | 2000 | Putative UDP-glucose-4-epimerase |
| ML1009 | cyp121 | rv2276 | +368 | 2000 | Cytochrome P450 |
| ML1006 | bcg3787 | rv3727 | +720 | 2000 | Putative oxidoreductase |
| Group C | | | | | |
| ML1025 | bcg3145 | rv3124 | +435 | 250 | Putative transcriptional regulator |
| Group D | | | | | |
| ML1065 | bcg0061 | rv0030 | +96 | >2000 | Unknown |
| ML1048 | bcg1567c | rv1503c | +519 | 250 | Unknown; survival in macrophages |
| ML1030 | bcg1988c | rv1949c | +849 | 2000 | Unknown; LAM synthesis |
| ML1053 | bcg2326c | rv2307B | +271 | 2000 | Unknown |
| ML1029 | bcg2734c | rv2721c | +524 | >2000 | Putative conserved transmembrane Ala/Gly- |
| | | | | | rich protein |
| ML1038 | bcg2735 | rv2722 | +78 | 2000 | Unknown |
| ML1046 | bcg2743 | rv2730 | +33 | 500 | Unknown |
| ML1052 | bcg2824 | rv2806 | +78 | >500 | Unknown |
| ML1050 | bcg2827 | rv2809 | +41 | 2000 | Unknown |
| ML1037 | bcg3693 | rv3635 | +1765 | 250 | Unknown |
| ML1012 | bcg3960c | rv3903c | +1255 | >2000 | Unknown |
| Group E | | | | | |
| ML1034 | bcg0231 | rv0194 | −54 | 2000 | ABC transporter |
| ML1060 | fadD25/ | rv1521/ | −206 | 250 | Fatty acid-conenzyme A ligase/conserved |
| | mmpL12 | rv1522c | | | transmembrane protein |
| ML1062 | pks11 | rv1665 | −18 | >2000 | Chalcone synthase |
| ML1044 | ppe33b | rv1810 | −178 | 250 | PPE family protein |
| ML1042 | bcg2123c/ | rv2104c/ | −509 | 500 | Putative transposase/PE family protein |
| | pe22 | rv2107 | | | |
| ML1051 | bcg2965 | rv2943 | −420 | >500 | Probable transposase |
| ML1005 | mmpL8 | rv3823c | −76 | ND | Conserved transmembrane protein; sulfolipid |
| | | | | | transport |

### Sequence Analysis and Functional Classification of Ampicillin-Resistant Transposon Mutants.

To determine the insertion sites of the transposon, chromosomal DNA was prepared from all mutants and was analyzed by ligation-mediated PCR (Prod'hom et al., FEMS Microbiol. Lett. 158:75-81 (1998)). Thirty-three unique insertion sites in M. bovis BCG were determined that conferred a medium or high level of resistance to ampicillin (Table 5). The mutants were grouped into four functional classes based on the predicted or known functions of the disrupted genes (Table 5). The vast majority of the disrupted genes (11/31) encode proteins involved in cell wall biosynthesis or assembly (Table 5, group A). Other mutant classes included genes involved in general metabolism and genes of unknown function. Six of the sequenced mutants had insertions in intergenic regions (Table 5).

### The Mutant ML1034 is Highly Resistant to Multiple Drugs.

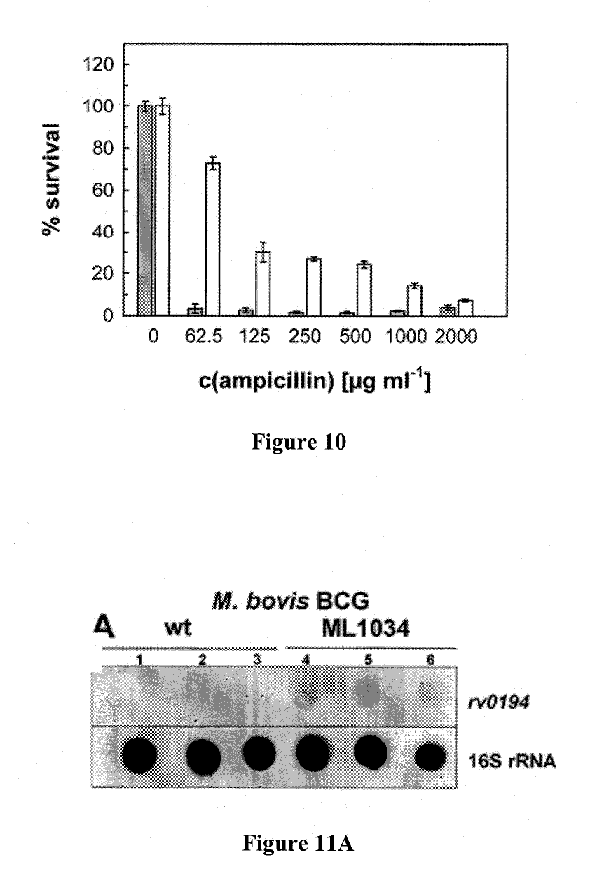

In the ML1034 mutant, the transposon had inserted 54 base pairs in front of the predicted start codon of the open reading frame bcg0231 in M. bovis BCG, which is almost identical to rv0194 from M. tuberculosis (Table 5; FIG. 9). Blast analysis revealed that rv0194 encodes a putative ATP-binding cassette (ABC) transporter. The Rv0194 protein consists of two membrane-spanning domains, each consisting of six predicted transmembrane helices and two cytoplasmic nucleotide-binding domains fused together. Hence, Rv0194 constitutes a complete multidrug efflux pump (Braibant et al., FEMS Microbiol. Rev. 24:449-67 (2000)). However, the function of this protein has not been demonstrated experimentally. The bcg0231 and rv0194 genes differ only by one base pair which causes a proline-toleucine exchange at position 328. This amino acid is located in one of the cytoplasmic loops, is not part of known functional domains of ABC transporters, and should, therefore, not cause any functional difference. The mutant ML1034 was extremely resistant to ampicillin, with its MIC increased by 32-fold from 62.5 μg/ml to 2,000 μg/ml (FIG. 10).

To examine whether the resistance of this mutant was specific for ampicillin, its sensitivity to several structurally unrelated antibiotics was determined in a MABA (Franzblau et al., J. Clin. Microbiol. 36:362-6 (1998)) (Table 6). The resistance to the small antibiotics chloramphenicol and tetracycline was 64- and more than 8-fold increased, respectively, compared to wt M. bovis BCG. Resistance to streptomycin, one of the first-line drugs used for the treatment of tuberculosis, was increased by 32-fold compared to wt M. bovis BCG. A fourfold increase in the resistance to the large and hydrophilic antibiotic vancomycin was also observed for the ML1034 mutant (Table 6), therefore suggesting that this mutant is highly resistant to multiple antibiotics.

TABLE 6
Susceptibility of wild-type M. bovis BCG and the ML1034 mutant.

| | MIC (μg/ml) | MIC (μg/ml) | Resistance |
|---|---|---|---|
| Antibiotic | WT | ML1034 | Factor |
| Ampicillin | 62.5 | 2000 | 32 |
| Chloramphenicol | 8 | 512 | 64 |
| Streptomycin | 0.5 | 16 | 32 |
| Tetracyclin | 2 | >16 | >8 |
| Vancomycin | 5 | 20 | 4 |

Insertion of the Transposon Increases Transcription of bcg0231 in the ML1034 Mutant.

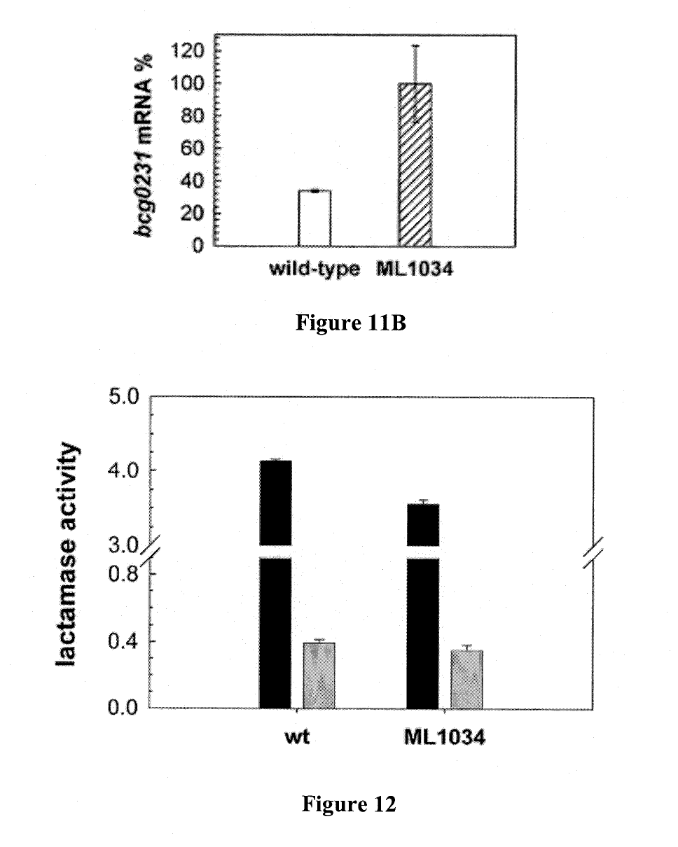

In case of insertion of the transposon in front of a gene, two possibilities exist: the transposon can inactivate the gene by inactivating the promoter or other signals required for transcription, or alternatively, gene expression can be upregulated or the gene can be expressed constitutively from a promoter inside the transposon. To distinguish between these two possibilities, total RNA was prepared from wt M. bovis BCG and the ML1034 mutant grown to late logarithmic phase. Dot blot experiments were used to quantify the relative amount of bcg0231 mRNA in both strains. While bcg0231 mRNA was barely detectable in wt M. bovis BCG, it was clearly more visible in the ML1034 mutant (FIG. 11A). Quantitative image analysis showed a threefold increase in the amount of bcg0231 mRNA relative to the 16S rRNA in the ML1034 mutant (FIG. 11B). The lane profile across each dot revealed that the signal for 16S rRNA was not saturated in samples 3 and 6 (FIG. 11A). Quantification of these signals revealed an 8.5-fold-increased amount of bcg0231 mRNA in the ML1034 mutant. In conclusion, these results demonstrated that insertion of the transposon increased transcription of the bcg0231 gene in the ML1034 mutant by at least threefold. This result suggested that the ABC transporter Bcg0231 constitutes an efflux pump and its increased expression caused the multidrug resistance of the ML1034 mutant. A similar activation of gene expression by insertions in front of genes due to promoters inside the transposon has been observed previously, for example, for the mpr gene of M. smegmatis (Rubin et al., Proc. Natl. Acad. Sci. USA 96:1645-50 (1999)).

The β-Lactamase Activities of wt M. bovis BCG and the Mutant ML1034 are Identical.

To examine whether altered expression of β-lactamases contributed to the high resistance of the ML1034 mutant to ampicillin, the β-lactamase activity of M. bovis BCG was measured using the nitrocefin hydrolysis assay. The vast majority of the β-lactamase activity of wt M. bovis BCG was cell associated and was approximately 10-fold higher than the activity of the culture filtrate (FIG. 12). Importantly, the β-lactamase activity of the ML1034 mutant was not higher than in the wt strain. Therefore, the increased resistance of the ML1034 mutant to ampicillin is not caused by faster hydrolysis of the drug. This result is consistent with high resistance of ML1034 to ampicillin as a direct result of the overexpression of the Bcg0231 pump that reduced the accumulation of ampicillin inside the cell. It should be noted that the multidrug resistance of the ML1034 strain also strongly argues against secondary mutations as a cause of this phenotype, because such mutations are specific for a single antibiotic in most cases (Wright, Curr. Opin. Chem. Biol. 7:563-9 (2003)).

Rv0194 Confers Multidrug Resistance to M. smegmatis.

To examine whether the multidrug-resistant phenotype of the ML1034 mutant was directly associated with overexpression of the ABC transporter, the rv0194 expression vector pML655 was transformed into M. smegmatis SMR5 and M. bovis BCG. In several attempts, colonies were only obtained for M. smegmatis, and not for M. bovis BCG. Importantly, overexpression of rv0194 increased the MICs of ampicillin, vancomycin, novobiocin, and erythromycin for M. smegmatis (Table 7). Similar resistance factors were obtained when rv0194 was overexpressed in M. smegmatis mc²155. These results confirmed that the multidrug resistance of the ML1034 mutant was directly associated with overexpression of rv0194.

TABLE 7
Susceptibility of M. smegmatis overexpressing
the rv0194 gene of M. tuberculosis.

| | | MIC (μg/ml) | |
|---|---|---|---|
| | MIC (μg/ml) | rv0194 | Resistance |
| Antibiotic | WT | overexpression | Factor |
| Ampicillin | 125 | 250 | 2 |
| Chloramphenicol | 32 | 32 | 1 |
| Erythromycin | 2.5 | 10 | 4 |
| Novobiocin | 4 | 8 | 2 |
| Tetracyclin | 0.3 | 0.3 | 1 |
| Vancomycin | 1.25 | 2.5 | 2 |

Rv0194 Reduces Accumulation of Ethidium Bromide in M. smegmatis.

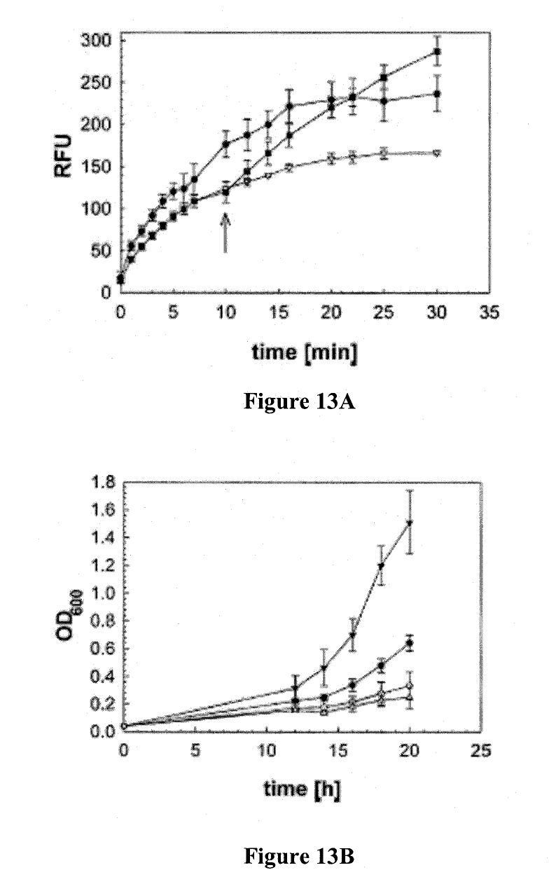

An obvious experiment to examine the function of Bcg0231/Rv0194 would be to measure accumulation of antibiotics whose MICs are increased drastically for the ML1034 mutant. However, uptake by slow-growing mycobacteria is very slow (Mailaender et al., Microbiology 150:853-64 (2004)), and interpretation of the uptake experiments is complicated by the high amount of surface-adsorbed compounds. This problem is even more pronounced for antibiotics which are substrates of drug efflux pumps (De Rossi et al., FEMS Microbiol. Rev. 30:36-52 (2006)). Therefore, ethidium bromide was chosen as a model compound whose uptake can be measured continuously. This assay is based on the increased fluorescence of ethidium bromide by binding to nucleic acids after entry into the bacterial cell (Margolles et al., Biochemistry 38:16298-306 (1999)) and does not, therefore, suffer from surface adsorption as all radiolabeled compounds. Importantly, expression of rv0194 reduced accumulation of ethidium bromide in M. smegmatis compared to the wt strain (FIG. 13). When reserpine, an inhibitor of multidrug transporters (Ahmed et al., J. Biol. Chem. 268:11086-9 (1993)), was added to the rv0194-expressing strain, accumulation of ethidium bromide quickly reached levels observed for wt M. smegmatis (FIG. 13A). Then, the influence of Rv0194 on the ability of M. smegmatis to grow in the presence of ethidium bromide was determined in order to examine whether the efflux activity of Rv0194 also increased the resistance of M. smegmatis. Growth of wt M. smegmatis and the rv0194-overexpressing strain in Middlebrook 7H9 medium did not differ. By contrast, addition of 1.56 μM ethidium bromide drastically reduced the growth of wt M. smegmatis compared to the rv0194-expressing strain (FIG. 13B). This was caused by an increase of the MIC of ethidium bromide from 1.25 μg/ml for the wt to 2.5 μg/ml for the pω0194-expressing strain. This effect was reversed by addition of 8 mM reserpine, which completely inhibited growth of both strains (FIG. 13B). These results demonstrate that Rv0194 directly extrudes ethidium bromide and that this activity increases the resistance of M. smegmatis to ethidium bromide. This clearly established the link between the efflux activity of Rv0194 and increased drug resistance of M. smegmatis and strongly suggests that the multidrug resistance of M. bovis BCG ML1034 is associated with increased efflux of antibiotics due to expression of Rv0194.

### Example 3

### M. smegmatis Ms3747 and M. tuberculosis Rv1698 are Involved in Copper Efflux

M. smegmatis Lacking Ms3747 is Hypersensitive to Copper Ions.

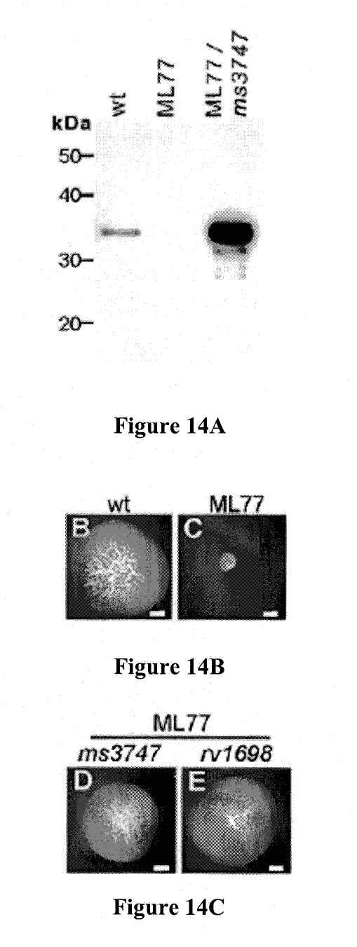

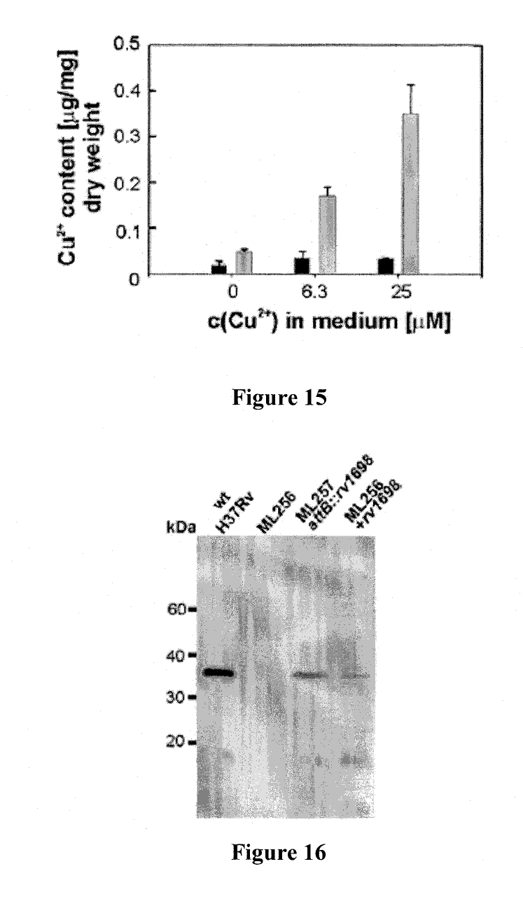

Rv1698 and its homolog M. smegmatis Ms3747 share 62% identical amino acids. To examine the physiological functions of these proteins, we inactivated the ms3747 gene in an unmarked mutant of M. smegmatis. Western blot experiments demonstrated the absence of Ms3747 protein in detergent extracts of ML77 (FIG. 4A). Expression of ms3747 and rv1698 from plasmids was 14-fold increased above wt levels. The average diameter of ML77 colonies was drastically decreased compared to the size of wt colonies on Middlebrook 7H10 plates (FIG. 14B). Expression of both ms3747 and rv1698 in ML77 complemented this phenotype (FIG. 14C) demonstrating that the growth defect of ML77 was caused by disruption of the ms3747 gene and not by secondary mutations, and that both genes have the same function. The ML77 strain grew as well as wild-type (wt) on LB plates and on self-made copper-free 7H10 medium (FIG. 15) indicating that ML77 has no general growth defect, but is more susceptible to copper. Growth of the ms3747 mutant was affected by four-fold lower copper concentrations than that of wt M. smegmatis. This demonstrated that ms3747 and rv1698 mediate resistance to copper. Silver ions were the only other metal ions tested to which ML77 was also more susceptible.

### The Outer Membrane (OM) Proteins Ms3747 and Rv1698 are Involved in Copper Efflux

To test the hypothesis that the OM channel protein Rv1698 of Mtb and its homolog Ms3747 of M. smegmatis are involved in copper efflux, copper accumulation in cells of wt M. smegmatis and the ms3747 mutant ML77 was examined. These strains were grown in the absence or presence of 6.3 or 25 μM copper in self-made 7H9 medium. Their copper content was analyzed after cell lysis by measuring the absorption of the Cu²′-dithizone complex as described (Kumar et al., Microchima Acta 105:79-87 (1991)). The copper content of wt M. smegmatis did not change regardless of the external Cu²⁺ concentration (FIG. 15). By contrast, the copper content of ML77 increased by 11-fold at 25 μM external Cu²⁺ (FIG. 15).

Rv1698 is not a General Porin of M. tuberculosis

Since Rv1698 partially complemented the slower uptake of glucose by a porin mutant of M. smegmatis, the permeability of wt M. smegmatis and ML77 for glucose was compared. No difference was observed for the uptake of 20 μM glucose at 37° C. by both strains demonstrating that the lack of ms3747 did not alter the OM permeability of M. smegmatis for this solute. This result is consistent with the fact that ML77 still expresses wt levels of the porin MspA, which is the major determinant of the OM permeability of M. smegmatis for hydrophilic solutes. It was concluded that Ms3747 and Rv1698 do not play a role in uptake, but are involved in efflux of copper. Efflux is a known copper resistance mechanism in other bacteria. These proteins have been named MctB (mycobacterial copper transport).

The mctB Mutant of M. tuberculosis is More Susceptible to Copper

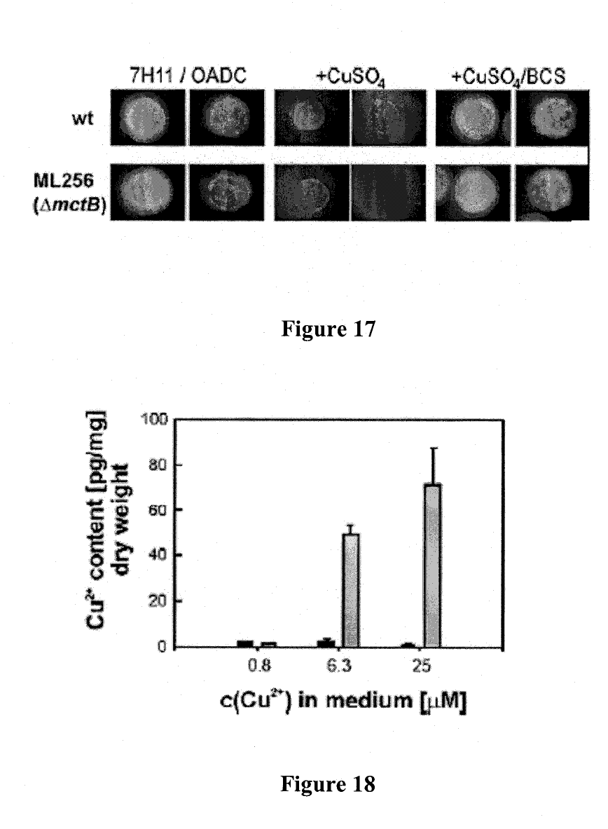

To examine the function of mctB in Mtb, unmarked mctB mutant ML256 was constructed and complemented with the mctB expression plasmid pML955 that integrates at the attB site of mycobacteriophage L5. Analysis of the SDS-extracts of these strains in a Western blot showed that wt Mtb expressed MctB_{Mtb}, the mctB_{Mtb} mutant ML256 did not, and integration of pML955 restored mctB_{Mtb} expression in the mutant (FIG. 16). To examine the role of MctB for the resistance of Mtb to copper, dilutions of cultures in a drop assay were used as described (Gold et al., Nat. Chem. Biol. 4:609-16 (2008)). Wt Mtb and the mctB mutant ML256 grew to the same colony size on reference Middlebrook 7H11 agar plates with OADC, likely because albumin binds to and neutralizes free copper in the media. Addition of 150 μM CuSO₄ severely reduced the growth rate of the mctB mutant in contrast to wt (FIG. 17). Bathocuproine disulfonate (BCS) binds Cu(I) and protects Mtb from the toxic effects of CuSO₄ indicating that Cu(I) is more toxic for the mctB mutant than for wt Mtb. The copper content of wt Mtb and ML256 cells was analyzed using the dithizone reagent as described for M. smegmatis (Kumar et al., Microchima Acta 105:79-87 (1991)). The amount of intracellular copper in wt Mtb was low and independent on external CuSO₄ concentrations (FIG. 18). By contrast, the copper content of ML256 increased drastically by 100-fold with increasing CuSO₄ concentrations in the medium (FIG. 18). These experiments demonstrated that MctB is required for maintaining a low cytoplasmic copper concentration in Mtb and for efficient resistance of Mtb to copper. The lack of the metallothionine MymT also increased the susceptibility of Mtb to copper. Taken together, these observations show that Mtb has at least two resistance mechanisms against copper, which are partially redundant, but can be overwhelmed by a drastic increase in external copper concentrations. It is concluded that the OM channel MctB is required for copper efflux in Mtb as is Ms3747 for M. smegmatis, demonstrating that these proteins are part of a novel mycobacterial copper efflux system. These results also show that copper efflux does not work without MctB underlining the importance of outer membrane proteins for transport processes in Mtb.

Copper as a Defensive Weapon of Macrophages Against M. tuberculosis

The minimal inhibitory concentration of copper for M. tuberculosis on Hartmans de Bond medium is less than 24 μM and much lower than that of E. coli (≈3 mM) or other bacteria (Franke et al., J. Bacteriol. 185:3804-12 (2003)). The extraordinary susceptibility of M. tuberculosis to copper appears surprising considering the extreme resistance of M. tuberculosis to many toxic solutes (Brennan and Nikaido, Annu Rev. Biochem. 64:29-63 (1995)). The high susceptibility of M. tuberculosis to copper probably has been overlooked because of the use of albumin in culture media in previous experiments (Ward et al., J. Bacteriol. 190:2939-46 (2008)) which sequesters copper ions (Suzuki et al., Arch. Biochem. Biophys. 273:572-7 (1989) and thereby strongly increases the tolerance of M. tuberculosis. Importantly, copper concentrations of 17 to 25 μM have been determined in M. tuberculosis-containing phagosomes of macrophages by microprobe X-ray fluorescence (Wagner et al., J. Immunol. 174:1491-1500 (2005)). Further, phagosomal copper concentrations appeared to increase upon stimulation of the macrophages with IFN-γ. Thus, activated macrophages appear to deliver copper at concentrations sufficient to inhibit or kill M. tuberculosis. These findings suggest that macrophages may utilize copper as a defensive weapon against M. tuberculosis.

### MctB is Required for Virulence of Mtb in Mice

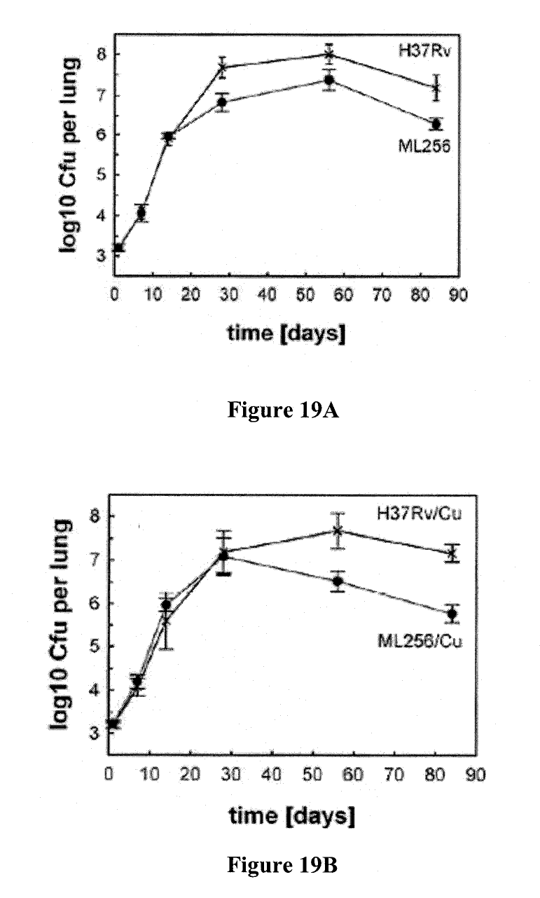

Fourty BALB/c mice per strain were infected in the aerosol chamber with Mtb H37Rv and the mctB mutant ML256. The inoculum was adjusted to implant 500-1000 bacteria in the lungs. Four mice per group were sacrificed on the day following infection. Then, four mice per group were sacrificed on weeks 1, 2, 4, 8, 12 and 16. Lungs were removed from the mice and the colony-forming units (CFU) were obtained by plating the appropriate dilutions of homogenized lungs on Middlebrook 7H11 agar plates. In the first two weeks after infection, the mctB mutant replicated as well as wt Mtb in mice (FIG. 19A). After the third week, 10-fold less bacterial cells of the mctB mutant were present in the lungs of mice compared to wt Mtb. This indicated that the mctB mutant has a persistence defect. In a second experiment, whether the increased dietary uptake of copper by mice would impair growth of the mctB mutant was investigated. Therefore, different CuSO₄ levels were tested in the drinking water to identify the highest CuSO₄ concentration that did not impair the health of mice. Then, a similar infection experiment was done as described above with 118 mg/L CuSO₄ in the drinking water. Again, a persistence defect of the mctB mutant was observed. The additional dietary uptake of CuSO₄ aggravated the persistence defect of the Mtb mctB mutant in mice (FIG. 19B). These results demonstrated that copper efflux by MctB is required for full virulence of Mtb in mice. Considering the 100-fold increased copper accumulation in the mctB mutant, it is concluded that the other copper resistance mechanisms in Mtb can be overwhelmed without a functional efflux system and that this impairs survival of Mtb in the persistence phase.

### Example 4

### Identification of Outer Membrane Efflux Channel of M. tuberculosis Existence of OM-Spanning Channel Proteins that Interact with Efflux Pumps in M. tuberculosis

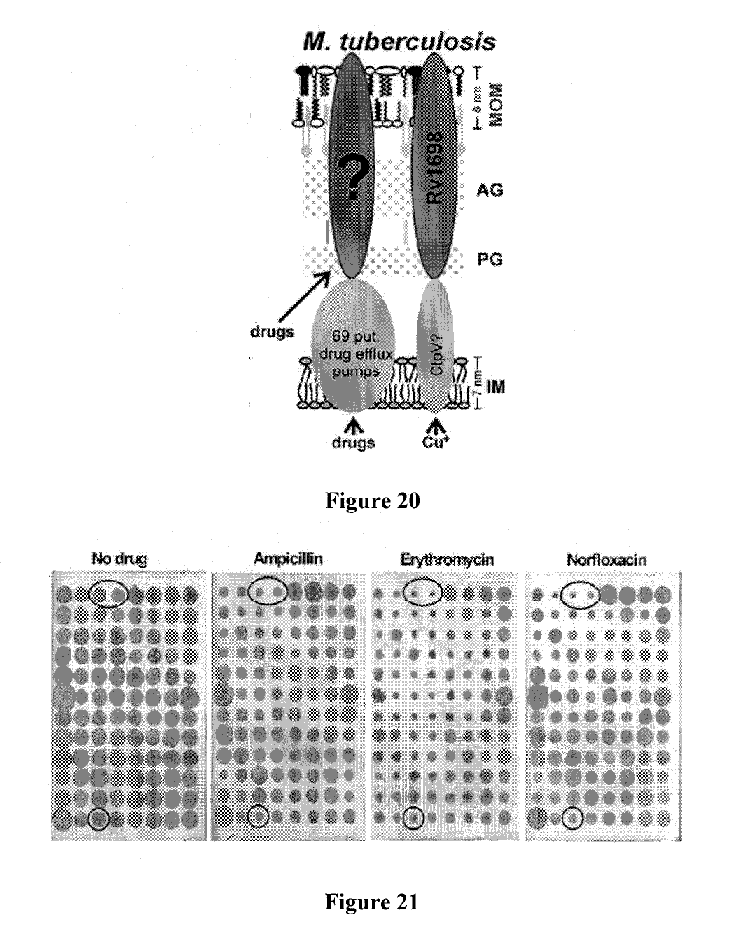

The existence in mycobacteria of such OM-spanning channel proteins that interact with efflux pumps has been recently demonstrated: Rv1698 (MctB) is an OM channel protein of Mtb (above) (Siroy et al., J. Biol. Chem. 283:17827-37 (2008)). Mutants of M. smegmatis and M. tuberculosis lacking MctB are more susceptible to copper because they accumulate large amounts of copper in contrast to the wild-type. OM channel proteins could in principle also be required for uptake of solutes. However, in such a case, the lack of these proteins would cause an increased resistance to the toxic effects of such compounds as shown for example for porin mutants. Therefore, these results indicate that MctB is part of a novel copper efflux system in mycobacteria (FIG. 20). Metal and drug-efflux systems share the same tripartite architecture in gram-negative bacteria. This indirect evidence strongly suggests the existence of an OM channel protein which is part of a multi-component drug-efflux system in Mtb.

### Screen of an Mtb Transposon Library for Mutants More Susceptible to Multiple Drugs

E. coli mutants which lack TolC are highly susceptible to many drugs because efflux through the tripartite systems has been abolished (Sulavik et al., Antimicrob. Agents Chemother. 45:1126-36 (2001)). Thus, screening of a mutant library for increased susceptibility to multiple drugs should yield clones that lack an essential part of important drug efflux systems such as a TolC-like protein. This assumption has been tested using an ordered library of 20,000 transposon mutants of M. smegmatis. An initial robotic screen yielded 398 clones that were susceptible to 8 μg/mL chloramphenicol in contrast to wt M. smegmatis. A second screen on agar plates showed that 27 out of these 398 clones were more susceptible than wt M. smegmatis to chloramphenicol, ampicillin, erythromycin and norfloxacin (FIG. 21). These findings were confirmed by the Alamar blue assay. Sequencing of the transposon insertion sites in nine selected clones revealed four genes whose involvement in drug resistance of M. smegmatis has not been shown before. Three genes encoded transporter proteins (Ms1683, Ms2927, Ms2737) and one protein involved in lipid biosynthesis (Ms5914). The predicted functions of these proteins are consistent with their involvement in either drug efflux or in the OM permeability barrier. However, none of those genes encoded a putative OM protein based on the secondary structure features shared by most OM proteins. The transposon library contained many duplicates and was, therefore, not comprehensive, thus explaining the lack of putative OM proteins being identified in the screen. Nevertheless, these experiments provided proof of principle that this screening approach yields multi-drug resistance genes. Thus, these findings enable one to use the same approach to screen for a TolC-like protein in Mtb.

A high-density mutant library of the avirulent Mtb mc26230 strain (ΔRD1 ΔpanCD) is constructed using a plasmid-based IS1096::Km transposon as published (Danilchanka et al., Antimicrob. Agents Chemother. 52:2503-11 (2008)). This library comprises approximately 20,000 mutants and is ordered in microplates and screened for clones with increased sensitivity to isoniazid, ethambutol, rifampicin, pyrazinamide, norfloxacin and erythromycin at half of their inhibitory concentration (IC50). These drugs are structurally diverse and are exported by drug efflux pumps of Mtb (DeRossi et al., FEMS Micrbiol. Rev. 30:36-52 (2006)).

The transposon library of the avirulent Mtb mc26230 strain is ordered into approximately 55 384-well plates, and frozen as glycerol stocks. On each 384-well plate 20 cultures of the parent strain are included as internal controls. All cultures are inoculated in fresh Middlebrook 7H9 liquid media, using a robotic liquid handler (Staccato ALH, Caliper Life Sciences). Each culture of these freshly grown ‘source’ is transferred to arrays and destination arrays are created that are comprised of agar media (a single slab of media, with the same external footprint of a micro-well plate; see FIG. 21 as an example for a 96 well plate). The media in the destination arrays contains the desired drug at IC50 concentrations. Prior to ‘printing’ each group of destination arrays, source cultures are resuspended by orbital shaking (aided by a submerged 384-pin tool) and diluted (by transfer to fresh media) if needed. During incubation at 37° C., the cell arrays are imaged using an Epson 10,000XL Scanner. Ten arrays are scanned simultaneously (˜2 minutes per scan) such that 200 arrays can be imaged in less than 30 minutes. The agar arrays are kept in a humidified incubator to prevent drying. Imaging is performed at least once per generation time (every 24 hours) for one month. Also implemented is a robotic imaging system. After four months screening is fully automated. Automated image analysis methods developed are used for growth curve analysis (Shah et al., BMC Syst. Biol. 1:3 (2007)). A database for storing and retrieving all associated data has been established, further facilitating quantitative analysis of growth rate differences with high sensitivity, allowing detection of subtle differences in drug sensitivity.

Crosslinking of Proteins that Interact with the Novel Drug Efflux Pump Rv0194 of Mtb

Overexpression of the recently discovered inner membrane drug efflux pump Rv0194 in M. bovis BCG confers complete resistance to ampicillin. Since the target of β-lactam antibiotics is in the periplasm, a mechanism must exist of how transport across the OM is coupled with the Rv0194 pump in the inner membrane. This strongly suggests that Rv0194 is connected with an OM channel protein. Two-hybrid systems are often used to identify protein-protein interactions. However, these systems are based on interactions of protein fragments which assemble to a cytoplasmic protein. Therefore, they are not useful for analyzing interactions of OM proteins. Direct purification of protein complexes and analysis of their composition appears to be difficult because solubilization by detergents often leads to dissociation of interacting proteins. Therefore, in vivo crosslinking experiments and mass spectroscopy are employed to identify proteins that interact with Rv0194. This is an alternative approach to identify a TolC-like protein of Mtb.

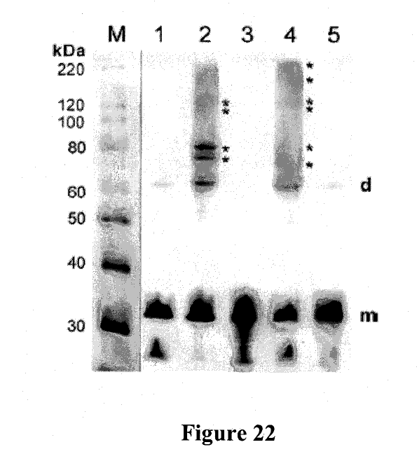

Crosslinking experiments have been done with the novel OM channel protein MctB, which most likely connects to an IM copper efflux pump in a protein complex, which, at least in Gram-negative bacteria, is very similar to the TolC-containing drug efflux system which spans two membranes (Li and Nikaido, Drugs 64:159-204 (2004); Murakami et al., Nature 419:587-593 (2002)). This novel copper efflux system of Mtb provides a paradigm for the drug efflux system. Crosslinking in whole cells of M. smegmatis revealed that MctB forms several complexes with other proteins (FIG. 22). This not only indicates that the OM copper channel MctB interacts with other proteins, but also shows that the alternative approach to identify a TolC-like protein in mycobacteria is feasible.

To identify proteins that interact with Rv0194, water-soluble and hydrophobic, membrane-permeable cross-linking reagents including formaldehyde, Dithiobis (succinimidyl) propionate (DSP), 1-Ethyl-3-[3-dimethylaminopropyl]carbodiimide hydrochloride (EDC), and 3,3″-Dithiobis[sulfosuccinimidylpropionate] (DTSSP) are screened. First, a combined histidine/HA tag is added to the C-terminus of Rv0194 in order to purify and identify crosslinked protein complexes. Then, in vivo cross-linking reactions are performed using different reagents in an Mtb strain which expresses the Rv0194His/HA protein following a published protocol (Husain et al., J. Bacteriol. 186:8533-6 (2004)). After crosslinking, membrane proteins are solubilized by detergents and analyzed in Western blots using an HA antibody. A comparison of the electrophoretic mobility with that of the Rv0194His/HA protein labeled in vitro using the same cross-linking reagents reveals whether Rv0194His/HA is bound by other proteins. Crosslinked proteins are purified by exploiting the His-tag of Rv0194His/HA by Ni²⁺ affinity chromatography. The protein complexes are cleaved by trypsin. The peptides of crosslinked proteins are identified by mass fingerprinting using a top-of-the-line Fourier transform ion cyclotron resonance mass spectrometer (FT-ICR-MS; Thermo-Finnegan LTQ-FT). FTICR-MS has the ability to measure peptide masses at low ppm levels and also provides high mass accuracy and is, therefore, ideal to identify interactions of low-abundance proteins.

What is claimed is:

1. A method of reducing drug resistance in a Mycobacterium tuberculosis (Mtb) comprising contacting the Mtb with an agent, wherein the agent inhibits the activity of an efflux complex.

2. The method of claim 1, wherein the efflux complex comprises an efflux channel and an efflux pump.

3. The method of claim 2, wherein the efflux channel comprises Rv1698.

4. The method of claim 2, wherein the efflux channel comprises a TolC-like efflux channel.

5. The method of claim 2, wherein the efflux pump comprises Rv0194.

6. The method of claim 1, wherein the efflux complex comprises a TolC-like efflux channel and Rv0194.

7. The method of claim 2, wherein the agent inhibits the activity of the efflux channel.

8. The method of claim 7, wherein the agent is an efflux channel inhibitor or blocker.

9. The method of claim 8, wherein the efflux channel inhibitor or blocker comprises Ru(II)quaterpyridinium complex or a derivative thereof.

10. The method of claim 2, wherein the agent inhibits the activity of the efflux pump.

11. The method of claim 1, wherein the agent is selected from group consisting of a small molecule, a polypeptide, a nucleic acid, or a peptidomimetic.

12. A method of treating Mycobacterium tuberculosis (Mtb) in a subject, the method comprising:
(a) administering to the subject an agent that inhibits the activity of an efflux complex; and
(b) administering to the subject a tuberculosis treating agent.

13. The method of claim 12, wherein the efflux complex comprises an efflux channel and an efflux pump.

14. The method of claim 13, wherein the efflux channel comprises Rv1698.

15. The method of claim 13, wherein the efflux channel comprises a TolC-like efflux channel.

16. The method of claim 13, wherein the efflux pump comprises Rv0194.

17. The method of claim 12, wherein the efflux complex comprises a TolC-like efflux channel and Rv0194.

18. The method of claim 12, wherein the tuberculosis treating agent comprises an antibiotic.

19. The method of claim 13, wherein the agent inhibits the activity of the efflux channel.

20. The method of claim 19, wherein the agent is an efflux channel inhibitor or blocker.

21. The method of claim 20, wherein the efflux channel inhibitor or blocker comprises Ru(II)quaterpyridinium complex or a derivative thereof.

22. The method of claim 13, wherein the agent inhibits the activity of the efflux pump.

23. The method of claim 12, wherein the agent is selected from group consisting of a small molecule, a polypeptide, a nucleic acid, or a peptidomimetic.

24. A method of screening for an agent that reduces drug resistance in Mycobacterium tuberculosis (Mtb), the method comprising
(a) providing a Mtb with a mutant efflux complex; and
(b) contacting the Mtb with an agent to be tested and a tuberculosis treating agent, wherein reduced resistance to the tuberculosis treating agent in the presence of the agent to be tested, as compared to a control, indicates the agent to be screened reduces drug resistance in Mtb.

25. The method of claim 24, wherein the mutant efflux complex comprises a mutant efflux channel.

26. The method of claim 25, wherein the mutant efflux channel comprises Rv1698.

27. The method of claim 25, wherein the mutant efflux channel comprises a TolC-like efflux channel.

28. The method of claim 24, wherein the mutant efflux complex comprises a mutant efflux pump.

29. The method of claim 28, wherein the mutant efflux pump comprises Rv0194.

30. The method of claim 24, wherein the mutant efflux complex comprises a mutant efflux channel and a mutant efflux pump.

31. The method of claim 30, wherein the mutant efflux channel comprises a TolC-like efflux channel and the mutant efflux pump comprises Rv0194.

32. The method of claim 24, wherein the tuberculosis treating agent comprises an antibiotic.

33. The method of claim 24, wherein the agent to be tested is selected from the list comprising a small molecule, a polypeptide, a nucleic acid, or a peptidomimetic.


SEQUENCE LISTING (25 sequences)

SEQ ID NO: 1 - DNA, 218 bp, Mycobacterium tuberculosis
tctgcccgaaagggaaaacccggtgtcctttctggttatcgaagtgaccggaatattcggtgccggcggcgcacacgcgagaatggatgccgcgcacgagtttatgcgcttgttcgggttctgcccgaaagggaagacttgatttcccgttagttcaaccaccgggtgatcggcgcactgaacgagaaaggatatggcgaatgcgcacgaattgctgg

SEQ ID NO: 2 - DNA, 61 bp, Artificial Sequence
  Primer
gattacttaattaacagaaaggaggttaatatgatctcgttgcgtcaacatgcggtctcac

SEQ ID NO: 3 - DNA, 40 bp, Artificial Sequence
  Primer
atataatttaaatggaacacgccctaacgcgggcctactg

SEQ ID NO: 4 - DNA, 56 bp, Artificial Sequence
  Primer
cgttaattaagcagaaaggaggttaatctatgataacgctacgggcgcacgcgatc

SEQ ID NO: 5 - DNA, 44 bp, Artificial Sequence
  Primer
atataatttaaatgcgcctctactgcgggaccgtcaccgaagac

SEQ ID NO: 6 - DNA, 52 bp, Artificial Sequence
  Primer
aaatggactagtggtggtggtggtggtgctgggaaaccgtgactgacatcgc

SEQ ID NO: 7 - DNA, 20 bp, Artificial Sequence
  Primer
cgttctcggctcgatgatcc

SEQ ID NO: 8 - DNA, 37 bp, Artificial Sequence
  Primer
atatacatatggatactttgctgtccagcttgcgtag

SEQ ID NO: 9 - DNA, 37 bp, Artificial Sequence
  Primer
cattagccatggatactttgctgtccagcttgcgtag

SEQ ID NO: 10 - DNA, 37 bp, Artificial Sequence
  Primer
cattagcatatggataacgttctcggctcgatgatcc

SEQ ID NO: 11 - DNA, 20 bp, Artificial Sequence
  Primer
ttgctgtccagcttgcgtag

SEQ ID NO: 12 - DNA, 18 bp, Artificial Sequence
  Primer
aggcgatgccgagcaggt

SEQ ID NO: 13 - DNA, 45 bp, Artificial Sequence
  Primer
cggcatgcagaaaggaggttaattaatgaaggcaatcagtcgggt

SEQ ID NO: 14 - DNA, 23 bp, Artificial Sequence
  Primer
taatgcgcacgaattgctggtgg

SEQ ID NO: 15 - DNA, 28 bp, Artificial Sequence
  Primer
gcaagcttgtcaactcgccacccattcg

SEQ ID NO: 16 - DNA, 24 bp, Artificial Sequence
  Primer
tagcttattcctcaaggcacgagc

SEQ ID NO: 17 - DNA, 26 bp, Artificial Sequence
  Primer
gaggcggcagaaagtcgtcaggtcag

SEQ ID NO: 18 - DNA, 21 bp, Artificial Sequence
  Primer
caacgtgcgagtcacgctgtc

SEQ ID NO: 19 - DNA, 28 bp, Artificial Sequence
  Primer
cttctgcagcaacgccaggtccacactg

SEQ ID NO: 20 - DNA, 20 bp, Artificial Sequence
  Primer
ggcaaatccacgttggcgtc

SEQ ID NO: 21 - DNA, 44 bp, Artificial Sequence
  Primer
ctaatacgactcactatagggagacggcagaggtcgggtcgtcc

SEQ ID NO: 22 - DNA, 21 bp, Artificial Sequence
  Primer
tgctacaatggccggtacaaa

SEQ ID NO: 23 - DNA, 44 bp, Artificial Sequence
  Primer
ctaatacgactcactatagggagacgcttccggtacggctacct

SEQ ID NO: 24 - DNA, 20 bp, Artificial Sequence
  Primer
cgaaggtcagcgggtgctca

SEQ ID NO: 25 - DNA, 20 bp, Artificial Sequence
  Primer
ctcaccgaggcagttccata